(19) United States
(12) Patent Application Publication
(10) Pub. No.: US 2025/0034347 A1
(43) Pub. Date: Jan. 30, 2025

(54) USE OF ESTERASES

(71) Applicant: CARBIOS, CLERMONT-FERRAND (FR)

(72) Inventors: ALAIN MARTY, TOULOUSE (FR); GRÉGORY ARNAL, TOULOUSE (FR); SOPHIE DUQUESNE, TOULOUSE (FR); VINCENT TOURNIER, TOULOUSE (FR); ALEXANDRE GILLES, TOULOUSE (FR)

(21) Appl. No.: 18/710,760

(22) PCT Filed: Nov. 15, 2022
(86) PCT No.: PCT/EP2022/082016

§ 371 Date: May 16, 2024

(30) Foreign Application Priority Data
Nov. 16, 2021 (EP) 21306591.5

(51) Int. Cl. C08J 11/10; C12N 9/18

(52) U.S. Cl. CPC C08J 11/105; C12N 9/18; C08J 2367/02

(57) ABSTRACT

The present invention relates to the use of specific enzymes, more particularly of specific esterases having a polyester degrading activity, for the degradation of polyester or polyester containing material in acidic conditions at a pH between 3 and 6. The present invention also relates to a process for degrading polyester or polyester containing material, such as plastic products, comprises a step of enzymatic depolymerization implemented in acidic conditions at a pH between 3 and 6. The process of the invention is particularly suited to degrade polyethylene terephthalate, and polyethylene terephthalate containing material.

The present invention relates to the use of specific enzymes, more particularly of specific esterases having a polyester degrading activity, for the degradation of polyester or polyester containing material in acidic conditions, at a pH between 3 and 6. The present invention also relates to a process for degrading polyester or polyester containing material, such as plastic products comprising a step of enzymatic depolymerization implemented in acidic conditions, at a pH between 3 and 6. The process of the invention is particularly suited to degrade polyethylene terephthalate, and polyethylene terephthalate containing material.

### BACKGROUND

Plastics are inexpensive and durable materials, which can be used to manufacture a variety of products that find uses in a wide range of applications (food packaging, textiles, etc.). Therefore, the production of plastics has increased dramatically over the last decades. Moreover, most of them are used for single-use disposable applications, such as packaging, agricultural films, disposable consumer items or for short-lived products that are discarded within a year of manufacture. Because of the durability of the polymers involved, substantial quantities of plastics are piling up in landfill sites and in natural habitats worldwide, generating increasing environmental problems. For instance, in recent years, polyethylene terephthalate (PET), an aromatic polyester produced from terephthalic acid and ethylene glycol, has been widely used in the manufacture of several products for human consumption, such as food and beverage packaging (e.g.: bottles, convenience-sized soft drinks, pouches for alimentary items) or textiles, fabrics, rugs, carpets, etc.

Different solutions, from plastic degradation to plastic recycling, have been studied to reduce environmental and economic impacts correlated to the accumulation of plastic waste. Mechanical recycling technology remains the most-used technology, but it faces several drawbacks. Indeed, it requires an extensive and costly sorting and it leads to downgrading applications, due to an overall loss of molecular weight during the process and uncontrolled presence of additives in the recycled products. The current recycling technologies are also expensive. Consequently, recycled plastic products are generally non-competitive compared to virgin plastic.

Recently, innovative processes of enzymatic recycling of plastic products have been developed and described (e.g., WO 2014/079844, WO 2015/097104, WO 2015/173265, WO 2017/198786, WO 2020/094661, WO 2020/094646, and WO 2021/123299). Contrary to traditional recycling technologies, such enzymatic depolymerization processes cast off expensive sorting and allow the recovery of the chemical constituents of the polymer (i.e., monomers and/or oligomers). The resulting monomers/oligomers may be recovered, purified and used to re-manufacture plastic items with equivalent quality to virgin plastic items, so that such processes lead to an infinite recycling of plastics. These processes are particularly useful for recovering terephthalic acid and ethylene glycol from plastic products comprising PET. In these processes, the production of said monomers and/or oligomers, and in particular the production of terephthalic acid, decreases the pH of the reaction medium which may be detrimental for the degrading enzyme activity. To maintain the pH and thereby an optimum enzyme activity, bases are massively added to the reaction medium. In addition, the recovering of terephthalic acid by precipitation requires the use of strong acid, leading to a huge production of salts, hardly valuable. Accordingly, the use of bases and acids as well as the lack of valorisation of the salts significantly impact the cost of these processes.

By working on these issues, the inventors have identified specific enzymes able to degrade efficiently polyester in acidic conditions. The inventors have discovered that said enzymes may be used in acidic conditions, while maintaining a depolymerization yield satisfactory from economical and industrial point of view. Thereby, the need of base during the enzymatic depolymerization is greatly decreased, and no or few salts are produced.

### SUMMARY OF THE INVENTION

The present invention relates to the use of particular polyester degrading enzymes, derived from the amino acid sequence as set forth in SEQ ID No 1 under particular conditions. The amino acid sequence set forth in SEQ ID No 1 corresponds to the amino acids 36 to 293 of the amino acid sequence of the metagenome-derived cutinase described in Sulaiman et al., Appl Environ Microbiol. 2012 March, and is referenced G9BY57 in SwissProt.

In this regard, the invention relates to the use of an esterase for degrading a polyester or a polyester containing material, wherein the polyester or polyester containing material is contacting, at a pH between 3 and 6, with an enzyme having a polyester degrading activity at a pH between 3 and 6, and preferably at least at a pH comprised between 5 and 5.5, and wherein the enzyme (i) has at least 75%, 80%, 85%, 90%, 95%, 96%, 97%, 98% or 99% identity to the full length amino acid sequence set forth in SEQ ID No 1, and (ii) contains at least one substitution selected from T11E/M, R12D/N/Q/E/F, S13E, A14E/D, T16E, A17T, T61S/V, A62D, F90A/Y, Y92G/D, W155A, T157S, P179D/E, Q182D/E, F187Y/I, D203C/K/R, N204D/E/G, A205D, S206D/E, F208M/W/G/N/R/I/A/Q/L/S/T/E, N211D/Y/E, S212F, N213P/D, N214D, A215N, S218A, V219I, Y220M/F, Q237D, F238E, N241E/D, N243E/D, L247T, V170I, G135A, V167Q/T, S248C and A24R, wherein the positions are numbered by reference to the amino acid sequence set forth in SEQ ID No 1.

Preferably, the enzyme comprises at least one substitution selected from A14E/D, F90A/Y, Y92G/D, Q182D/E, N204D/E/G, F208M/W/G/N/R/I/A/Q/L/S/T/E, N211D/Y/E, N213P/D, V219I, V170I, D203C/K/R, S248C, A24R and F187Y/I, preferably selected from A14E, F90A, Y92G/D, Q182D/E, N204G, F208M/G/N/R/I/A/Q/L/S/T, N211D/E, N213P, V219I, V170I, D203C/K/R, S248C, A24R and F187I, more preferably selected from F90A, Y92G, Q182D/E, N204G, F208M/G/N/R/I/A/Q/L/S/T, N211D/E, V170I, D203C/K/R, S248C, N213P, A24R and F187I, even more preferably selected from N211D/E, N204G, F208M/R/I/Q/L/S/T, Q182D/E, Y92G, V170I, D203C/K/R, S248C, N213P, A24R and F187I.

Preferably, the esterase comprises at least one combination of substitution selected F208I+D203C+S248C+V170I+Y92G+N213P+Q182E, F208M+D203C+S248C+V170I+Y92G+N213P+Q182E, F208T+D203C+S248C+V170I+Y92G+N213P+Q182E, F208L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L and F208M+D203C+S248C+V170I+Y92G+N213P+Q182E+D158E, more preferably at least one combination of substitutions selected from F208M+D203C+S248C+V170I+Y92G+N213P+Q182E, F208I+D203C+S248C+V170I+Y92G+N213P+Q182E and F208M+D203C+S248C+V170I+Y92G+N213P+Q182E+S13L+D158E.

It is also an object of the invention to provide a process for degrading a polyester or polyester containing material comprising contacting said polyester or said polyester containing material with an esterase able to degrade said polyester, as described above, wherein the step of contacting the polyester or the polyester containing material with the enzyme is performed in acidic conditions, particularly at a pH between 3 and 6, preferably at a pH between 5 and 5.5.

The present invention also relates to a detergent composition comprising an esterase.

The present invention also relates to a polyester containing material comprising an esterase.

### DETAILED DESCRIPTION OF THE INVENTION

### Definitions

The present disclosure will be best understood by reference to the following definitions. Herein, the terms “peptide”, “polypeptide”, “protein”, “enzyme” refer to a chain of amino acids linked by peptide bonds, regardless of the number of amino acids forming said chain. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

The term “esterase” refers to an enzyme which belongs to a class of hydrolases classified as EC 3.1.1 according to Enzyme Nomenclature that catalyzes the hydrolysis of esters into an acid and an alcohol. The term “cutinase” or “cutin hydrolase” refers to the esterases classified as EC 3.1.1.74 according to Enzyme Nomenclature that are able to catalyse the chemical reaction of production of cutin monomers from cutin and water.

The terms “wild-type protein” or “parent protein” refer to the non-mutated version of a polypeptide as it appears naturally. In the present case, the parent esterase refers to the esterase having the amino acid sequence as set forth in SEQ ID No 1.

The terms “mutant” and “variant” refer to polypeptides derived from SEQ ID No 1 and comprising at least one modification or alteration, i.e., a substitution, insertion, and/or deletion, at one or more (e.g., several) positions as compared to SEQ ID No 1, and having a polyester degrading activity. The variants may be obtained by various techniques well known in the art. In particular, examples of techniques for altering the DNA sequence encoding the wild-type protein, include, but are not limited to, site-directed mutagenesis, random mutagenesis and synthetic oligonucleotide construction. Thus, the terms “modification” and “alteration” as used herein in relation to a particular position means that the amino acid in this particular position has been modified compared to the amino acid in this particular position in the wild-type protein.

A “substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues (G, P, A, V, L, I, M, C, F, Y, W, H, K, R, Q, N, E, D, S and T). The sign “+” indicates a combination of substitutions. In the present document, the following terminology is used to designate a substitution: L82A denotes that amino acid residue (Leucine, L) at position 82 of the parent sequence is substituted by an Alanine (A). A121V/I/M denotes that amino acid residue (Alanine, A) at position 121 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

Unless otherwise specified, the amino acid positions disclosed in the present application are numbered by reference to the amino acid sequence set forth in SEQ ID No 1.

As used herein, the term “sequence identity” or “identity” refers to the number (or fraction expressed as a percentage %) of matches (identical amino acid residues) between two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=11, Gap extend=1.

A “polymer” refers to a chemical compound or mixture of compounds whose structure is constituted of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers). According to the invention, “oligomers” refer to molecules containing from 2 to about 20 monomers.

In the context of the invention, a “polyester containing material” or “polyester containing product” refers to a product, such as plastic product or plastic article, comprising at least one polyester in crystalline, semi-crystalline or totally amorphous forms. In a particular embodiment, the polyester containing material refers to any item made from at least one plastic material, such as plastic sheet, tube, rod, profile, shape, film, massive block, fiber, etc., which contains at least one polyester, and possibly other substances or additives, such as plasticizers, mineral or organic fillers. In another particular embodiment, the polyester containing material refers to a plastic compound, or plastic formulation, in a molten or solid state, suitable for making a plastic product. In another particular embodiment, the polyester containing material refers to textile, fabrics or fibers comprising at least one polyester. In another particular embodiment, the polyester containing material refers to plastic waste or fiber waste comprising at least one polyester. Particularly, the plastic article is a manufactured product, such as rigid or flexible packaging (bottle, trays, cups, etc.), agricultural films, bags and sacks, disposable items or the like, carpet scrap, fabrics, textiles, etc. The plastic article may contain additional substances or additives, such as plasticizers, minerals, organic fillers or dyes. In the context of the invention, the plastic article may comprise a mix of semi-crystalline and/or amorphous polymers and/or additives.

In the present description, the term “polyester(s)” encompasses but is not limited to polyethylene terephthalate (PET), polytrimethylene terephthalate (PTT), polybutylene terephthalate (PBT), polyethylene isosorbide terephthalate (PEIT), polylactic acid (PLA), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polyethylene naphthalate (PEN) and blends/mixtures of these polymers. Polyesters can also encompasses “polyolefin-like” polyesters, preferably “polyethylene-like” polyesters which correspond to polyolefin (preferably polyethylene) into which ester segments have been introduced (generally achieved by polycondensation of long-chain α,ω-difunctional monomers), as defined in Lebarbe et al. Green Chemistry Issue 4 2014.

The term “depolymerization”, in relation to a polymer or plastic article containing a polymer, refers to a process by which the polymer or at least one polymer of said plastic article is depolymerized and/or degraded into smaller molecules, such as monomers and/or oligomers and/or any degradation products.

According to the invention, “oligomers” refer to molecules containing from 2 to about 20 monomer units. As an example, oligomers retrieved from PET include methyl-2-hydroxyethyl terephthalate (MHET) and/or bis(2-hydroxyethyl) terephthalate (BHET) and/or 1-(2-hydroxyethyl) and/or 4-methyl terephthalate (HEMT) and/or dimethyl terephthalate (DMT).

### Uses of Specific Esterases

By working on the optimisation of enzymatic degrading process of plastic products in acidic conditions, particularly at a pH between 3 and 6, and more particularly at a pH between 5 and 5.5, the inventors have identified specific esterases which exhibit an efficient polyester degrading activity, in particular a PET degrading activity, as well as an improved thermal stability in acidic conditions.

According to the present invention, “acidic conditions” refer to conditions (e.g., medium, solution, etc.) at a pH comprised between 3 and 6. Particularly, the “acidic conditions” refer to the conditions under which the esterases are used, e.g., the esterases are contacted with the polyester in a medium having a pH between 3 and 6 and more particularly in a medium having a pH between 5 and 5.5.

According to the present invention, the esterases exhibit an increased polyester degrading activity and/or an increased thermostability as compared to a parent, or wild-type esterase, having the amino acid sequence as set forth in SEQ ID No 1, in acidic conditions. Particularly, the esterases disclosed in the present invention exhibit an increased activity and/or an increased thermostability as compared to the parent esterase when submitted at a pH between 3 and 6. Accordingly, the increased activity and/or increased thermostability may be observed at specific pH between 3 and 6 and/or in a range of pH between 3 and 6. Particularly, the increased activity and/or increased thermostability may be observed at least at pH 3, at pH 3.5, at pH 4, at pH 4.5, at pH 5, at pH 5.2, at pH 5.5, and/or at pH 6. The increased activity and/or increased thermostability may also be observed in the whole range of pH 3 to 6, in the whole range of pH 4 to 6, in the whole range of pH 4.5 to 6, in the whole range of pH 5 to 6, in the whole range of pH 5.5 to 6, in the whole range of pH 5 to 5.5, in the whole range of pH 5 to 5.2, in the whole range of pH 5.2 to 5.5.

According to the invention, the esterases exhibit an increased activity at a pH comprised between 3 and 6, preferably at a pH between 4 and 6, more preferably at a pH between 5 and 6, even more preferably at a pH between 5 and 5.5, even more preferably at pH 5.2, compared to the esterase having the amino acid sequence as set forth in SEQ ID No 1, also referenced as parent esterase, at same pH.

Particularly, the inventors have identified specific amino acid substitutions to perform in SEQ ID No 1, which advantageously lead to an increased activity of the esterase on this polymer in acidic conditions.

Within the context of the invention, the term “increased activity” or “increased degrading activity” indicates an increased ability of the esterase to degrade a polyester and/or an increased ability to adsorb on a polyester, at given conditions (e.g., temperature, pH, concentration) as compared to the ability of the esterase of SEQ ID No 1 to degrade and/or adsorb on same polyester at same conditions. Such an increase may be at least 10% greater than the polyester degrading activity of the esterase of SEQ ID No 1, preferably at least 20%, 30%, 40%, 50%, 60%, 70%, 80%, 90%, 100%, 110%, 120%, 130% or greater. Particularly, the degrading activity is a depolymerization activity leading to monomers and/or oligomers of the polyester, which can be further retrieved and optionally reused. Particularly, the esterase of the invention has an increased PET degrading activity. Within the context of the invention, the esterase exhibits an increased degrading activity at least at a pH comprised between 3 and 6, as compared to the degrading activity of the parent esterase at same pH. Preferably, the esterase exhibits an increased activity at least at a pH comprised between 4 and 6, preferably between 5 and 6, more preferably between 5 and 5.5, even more preferably at pH 5.2.

The “degrading activity” of an esterase may be evaluated by the one skilled in the art, according to methods known per se in the art. For instance, the degrading activity can be assessed by measurement of the specific polymer's depolymerization activity rate, the measurement of the rate to degrade a solid polymer compound dispersed in an agar plate, or the measurement of the polymer's depolymerization activity rate in reactor. Particularly, the degrading activity may be evaluated by measuring the “specific degrading activity” of an esterase. The “specific degrading activity” of an esterase for PET corresponds to μmol of PET hydrolyzed/min or mg of equivalent TA produced/hour, and per mg of esterase during the initial period of the reaction (i.e. the first 24 hours) and is determined from the linear part of the hydrolysis curve of the reaction, such curve being set up by several samplings performed at different time during the first 24 hours. As another example, the “degrading activity” may be evaluated by measuring, after a defined period of time (for example after 24 h, 48 h or 72 h), the rate and/or yield of oligomers and/or monomers released under suitable conditions of temperature, pH and buffer, when contacting the polymer or the polymer-containing plastic product with a degrading enzyme.

The ability of an enzyme to adsorb on a substrate may be evaluated by the one skilled in the art, according to methods known per se in the art. For instance, the ability of an enzyme to adsorb on a substrate can be measured from a solution containing the enzyme and wherein the enzyme has been previously incubated with a substrate under suitable conditions.

The inventors have also identified target amino acid residues in SEQ ID No 1, that may be advantageously modified to improve the stability of corresponding esterases in acidic conditions at elevated temperatures (i.e., improved thermostability), particularly at a temperature at or above 50° C. and at or below 90° C., preferably at or above 60° C. and at or below 80° C., more preferably at or above 65° C. and at or below 75° C.

According to the present invention, the esterases exhibit an increased thermostability at a pH comprised between 3 and 6, preferably at a pH between 4 and 6, more preferably at a pH between 5 and 6, even more preferably at a pH between 5 and 5.5, even more preferably at pH 5.2, as compared to the thermostability of the esterase having the amino acid sequence set forth in SEQ ID No 1 (i.e., the parent esterase) at same pH.

Within the context of the invention, the term “increased thermostability” indicates an increased ability of an esterase to resist to changes in its chemical and/or physical structure at high temperatures, and particularly at temperature between 50° C. and 90° C., as compared to the esterase of SEQ ID No 1. Particularly, the thermostability of the esterases is improved, as compared to the thermostability of the parent esterase, at temperature between 50° C. and 90° C., between 50° C. and 80° C., between 50° C. and 75° C., between 50° C. and 70° C., between 50° C. and 65° C., between 55° C. and 90° C., between 55° C. and 80° C., between 55° C. and 75° C., between 55° C. and 70° C., between 55° C. and 65° C., between 60° C. and 90° C., between 60° C. and 80° C., between 60° C. and 75° C., between 60° C. and 70° C., between 60° C. and 65° C., between 65° C. and 90° C., between 65° C. and 80° C., between 65° C. and 75° C., between 65° C. and 70° C. Particularly, the thermostability of the esterases is improved, in acidic conditions, as compared to the thermostability of the parent esterase, at temperature(s) between 40° C. and 80° C., between 50° C. and 72° C., between 55° C. and 60° C., between 50° C. and 55° C., between 60° C. and 72° C. Preferably, the thermostability of the esterases is improved, as compared to the thermostability of the parent esterase, at least at temperature between 50° C. and 65° C. Within the context of the invention, temperatures are given at +/−1° C.

Particularly, the thermostability may be evaluated through the assessment of the melting temperature (Tm) of the esterase. In the context of the present invention, the “melting temperature” refers to the temperature at which half of the enzyme population considered is unfolded or misfolded. Typically, esterases of the invention show an increased Tm of about 0.8° C., 1° C., 2° C., 3° C., 4° C., 5° C., 10° C. or more, as compared to the Tm of the esterase of SEQ ID No 1 at a pH comprised between 3 and 6. In particular, at a pH comprised between 3 and 6, esterases of the present invention can have an increased half-life at a temperature between 50° C. and 90° C., as compared to the esterase of SEQ ID No 1. Particularly, esterases of the present invention can have an increased half-life at a temperature between 50° C. and 90° C., between 50° C. and 80° C., between 50° C. and 75° C., between 50° C. and 70° C., between 50° C. and 65° C., between 55° C. and 90° C., between 55° C. and 80° C., between 55° C. and 75° C., between 55° C. and 70° C., between 55° C. and 65° C., between 60° C. and 90° C., between 60° C. and 80° C., between 60° C. and 75° C., between 60° C. and 70° C., between 60° C. and 65° C., between 65° C. and 90° C., between 65° C. and 80° C., between 65° C. and 75° C., between 65° C. and 72° C., between 65° C. and 70° C., between 60° C. and 72° C., as compared to the esterase of SEQ ID No 1 at a pH comprised between 3 and 6. Advantageously, the esterases disclosed in the present invention have an increased half-life at least at a temperature between 50° C. and 65° C., as compared to the esterase of SEQ ID No 1 at a pH comprised between 3 and 6.

Within the context of the invention, the esterases disclosed in the present invention exhibit an increased thermostability as compared to the thermostability of the esterase having the amino acid sequence set forth in SEQ ID No 1, at least at a pH comprised between 3 and 6. Preferably, the esterase exhibits an increased thermostability at least at a pH comprised between 4 and 6, preferably between 5 and 6, more preferably between 5 and 5.5, even more preferably at pH 5.2.

The melting temperature (Tm) of an esterase may be measured by the one skilled in the art, according to methods known per se in the art. For instance, the DSF may be used to quantify the change in thermal denaturation temperature of the esterase and thereby to determine its Tm. Alternatively, the Tm can be assessed by analysis of the protein folding using circular dichroism. Preferably, the Tm is measured using DSF or circular dichroism as exposed in the experimental part. In the context of the invention, comparisons of Tm are performed with Tm that are measured under same conditions (e.g., pH, nature and amount of polyesters, etc.).

Alternatively, the thermostability may be evaluated by measuring the esterase activity and/or the polyester depolymerization activity of the esterase after incubation at different temperatures and comparing with the esterase activity and/or polyester depolymerization activity of the parent esterase. The ability to perform multiple rounds of polyester's depolymerization assays at different temperatures can also be evaluated. A rapid and valuable test may consist on the evaluation, by halo diameter measurement, of the esterase ability to degrade a solid polyester compound dispersed in an agar plate after incubation at different temperatures.

According to the invention, the esterases disclosed in the invention further exhibit a greater increase of polyester degrading activity and/or a greater increase of thermostability, compared to the enzyme of SEQ ID No 1 in acidic conditions than in basic conditions, i.e. above pH 7. Within the context of the present invention, “basic conditions” refer to conditions (e.g., medium, solution, etc.) at a pH above 7, preferably at a pH between 7 and 9.

Particularly, these esterases are more efficient and stable, comparatively to the parent esterase, at a pH between 3 and 6, particularly at a pH between 4 and 6, between 5 and 6, between 5 and 5.5, than at a pH of 7 or above 7, particularly at a pH between 7 and 9. Particularly, these esterases exhibit an increase of polyester degrading activity and/or an increase of thermostability compared to the enzyme of SEQ ID No 1 that is greater at a pH comprised between 3 and 6, particularly at a pH between 5 and 5.5, than at a pH of 7 or above 7, particularly at a pH between 7 and 9.

### Esterases to be Used

The present invention thus relates to the use of an esterase for degrading a polyester or a polyester containing material, wherein the polyester or polyester containing material is contacted, at a pH between 3 and 6, with an enzyme which (i) has at least 75%, 80%, 85%, 90%, 95%, 96%, 97%, 98% or 99% identity to the full length amino acid sequence set forth in SEQ ID No 1, (ii) contains at least one substitution selected from T11E/M, R12D/N/Q/E/F, S13E, A14E/D, T16E, A17T, T61S/V, A62D, F90A/Y, Y92G/D, W155A, T157S, P179D/E, Q182D/E, F187Y/I, D203C/K/R, N204D/E/G, A205D, S206D/E, F208M/W/G/N/R/I/A/Q/L/S/T/E, N211D/Y/E, S212F, N213P/D, N214D, A215N, S218A, V219I, Y220M/F, Q237D, F238E, N241E/D, N243E/D, L247T, V170I, G135A, V167Q/T, S248C and A24R, wherein the positions are numbered by reference to the amino acid sequence set forth in SEQ ID No 1 and (iii) has a polyester degrading activity at a pH comprised between 3 and 6.

Unless otherwise specified, the amino acid positions disclosed in the present application are numbered by reference to the amino acid sequence set forth in SEQ ID No 1

Advantageously, the esterase has a polyester degrading activity at a pH comprised between 4 and 6, preferably at a pH comprised between 5 and 6, more preferably at a pH comprised between 5 and 5.5, particularly at pH 5.2.

According to the invention, the esterase is contacted with the polyester or the polyester containing material at a pH comprised between 3 and 6, preferably between 4 and 6, more preferably between 5 and 6, even more preferably between 5 and 5.5, particularly at pH 5.2.

In an embodiment, the esterase comprises at least one substitution selected from 5 F208M/W/G/N/R/I/A/Q/L/S/T/E, T11E/M, R12D/N/Q/E/F, S13E, A14E/D, T16E, T61S/V, A62D, F90A/Y, Y92G/D, W155A, T157S, Q182D/E, D203C/K, N204E/G, S206D, N211D/Y/E, S212F, N213P, V219I, Y220M, Q237D, N241E/D, N243E, L247T, V170I, G135A, V167Q/T, S248C, A24R and F187I, preferably selected from F208M/W/G/N/R/I/A/Q/L/S/T/E, T11M, R12D/E/F, A14E/D, T61S/V, A62D, F90A/Y, Y92G, W155A, T157S, Q182D/E, D203C/K, N204E/G, N211D/Y/E, S212F, N213P, V219I, Q237D, N241E/D, V170I, G135A, V167Q/T, S248C, A24R and F187I.

In another embodiment, the esterase comprises at least one substitution selected from F208M/W/I/L/T/E, T11E/M, R12D/N/Q/E/F, S13E, A14E/D, T16E, T61S/V, A62D, F90A/Y, W155A, T157S, Q182D/E, N204E/G, S206D, N211D/Y, S212F, N213P, V219I, Y220M, Q237D, N241E/D, N243E and L247T, preferably selected from F208M/W/I/L/T/E, T11M, R12D/E/F, A14E/D, T61S/V, A62D, F90A/Y, W155A, T157S, Q182D/E, N204E, N211D/Y, S212F, N213P, V219I, Q237D and N241E/D.

Preferably, the esterase comprises at least one substitution selected from A14E/D, F90A/Y, Y92G/D, Q182D/E, N204D/E/G, F208M/W/G/N/R/I/A/Q/L/S/T/E, N211D/Y/E, N213P/D, V219I, V170I, D203C/K/R, S248C A24R and F187Y/I, preferably selected from A14E, F90A, Y92G/D, Q182D/E, N204G, F208M/G/N/R/I/A/Q/L/S/T, N211D/E, N213P, V219I, V170I, D203C/K/R, S248C A24R and F187I, more preferably selected from F90A, Y92G, Q182D/E, N204G, F208M/G/N/R/I/A/Q/L/S/T, N211D/E, V170I, D203C/K/R, S248C, N213P, A24R and F187I, even more preferably selected from N211D/E, N204G, F208M/R/I/Q/L/S/T, Q182D/E, Y92G, V170I, D203C/K/R, S248C, N213P A24R and F187I. For instance, the esterase comprises at least one substitution selected from F208T/L/S/R/G/I/M, Q182E, Y92G, V170I, D203C, S248C and N213P.

According to the invention, the esterase may comprise at least one substitution selected from N211D, N204G, F90A, F208M/G/N/R/I/A/Q/L/S/T, Q182D/E and A24R and F187I, preferably selected from N211D, N204G, F208M/R/Q/L/S/T, Q182D/E, A24R and F187I, more preferably selected from N211D, F208M/Q/L/S/T, Q182D/E, A24R and F187I.

According to the invention, the esterase may comprise at least the combination of substitutions Y92G+V170I+D203C+S248C and at least one substitution selected from N211D, N204G, F90A, F208M/G/N/R/IA/Q/L/S/T and Q182D/E, preferably selected from N211D, N204G, F208M/R/I/Q/L/S/T and Q182D/E, more preferably selected from F208M/I//L/T and Q182E.

According to the invention, the esterase may comprise at least the combination of substitutions Y92G+V170I+D203C+S248C+N213P+Q182E and at least one substitution selected from N211D, N204G, F90A and F208M/G/N/R/I/A/Q/L/S/T, preferably selected from N211D, N204G and F208M/R/I/Q/L/S/T, more preferably selected from N211D, N204G and F208M/I//L/T, even more preferably at least one substitution selected from F208M/I//L/T.

According to the invention, the esterase may comprise at least one substitution selected from F208M/W/G/N/R/I/A/Q/L/S/T/E, preferably selected from F208M/G/N/R/I/A/Q/L/S/T, more preferably selected from F208M/R/I/Q/L/S/T, even more preferably F208M/R/Q/L/S/T. Particularly, the esterase comprises at least one substitution selected from F208M/I/L/T. Particularly, the enzyme comprises at least the substitution F208M.

According to the invention, when the esterase comprises the substitution D203K/R, the amino acid residue S248 is preferably maintained, as in the parent esterase, i.e., the esterase of SEQ ID No 1.

According to the invention, the esterase may comprise at least two, preferably at least three, four, five, six, seven substitutions selected from A14E/D, F90A/Y, Y92G/D, Q182D/E, N204D/E/G, F208M/W/G/N/R/I/A/Q/L/S/T/E, N211D/Y/E, N213P/D, V219I, V170I, D203C/K/R, S248C A24R and F187Y/I, preferably selected from A14E, F90A, Y92G/D, Q182D/E, N204G, F208M/G/N/R/I/A/Q/L/S/T, N211D/E, N213P, V219I, V170I, D203C/K/R, S248C A24R and F187I, more preferably selected from F90A, Y92G, Q182D/E, N204G, F208M/G/N/R/I/A/Q/L/S/T, N211D/E, V170I, D203C/K/R, S248C, N213P A24R and F187I, even more preferably selected from N211D, N204G, F208M/R/IL/S/T, Q182D/E, Y92G, V170I, D203C/K/R, S248C, N213P A24R and F187I.

According to the invention, the esterase may comprise at least a combination of substitutions selected from F208M/W/G/N/R/I/A/Q/L/S/T/E+D203C+S248C+V170I+Y92G/D+N213P/D+Q182D/E and F208M/W/G/N/R/I/A/Q/L/S/T/E+D203K+V170I+Y92G/D+N213P/D+Q182D/E, preferably selected from F208M/I/L/T+D203C+S248C+V170I+Y92G+N213P+Q182E and F208M/I/L/T+D203K+V170I+Y92G/D+N213P/D+Q182D/E, more preferably selected from F208M/I/L/T+D203C+S248C+V170I+Y92G+N213P+Q182E and F208M/I/L/T+D203K+V170I+Y92G+N213P+Q182E.

For example, the esterase may comprise at least a combination of substitutions selected from F208M+D203C+S248C+V170I+Y92G+N213P+Q182E and F208M+D203K+V170I+Y92G+N213P+Q182E, preferably the combination of substitutions F208M+D203C+S248C+V170I+Y92G+N213P+Q182E.

According to the invention, the esterase may further comprise at least one substitution selected from S13L and D158E, preferably at least the two substitutions S13L and D158E.

For example, the esterase may comprise at least a combination of substitutions selected from F208M/I/L/T+D203C+S248C+V170I+Y92G+N213P+Q182E+S13L+D158E, F208M/I/L/T+D203C+S248C+V170I+Y92G+N213P+Q182E+S13L or F208M/I/L/T+D203C+S248C+V170I+Y92G+N213P+Q182E+D158E, preferably the combination F208M/I/L/T+D203C+S248C+V170I+Y92G+N213P+Q182E+S13L+D158E, more preferably the combination preferably the combination F208M+D203C+S248C+V170I+Y92G+N213P+Q182E+S13L+D158E.

In an embodiment, the esterase has the amino acid sequence set forth in SEQ ID No 1 with one to thirty-one substitutions selected from F208M/W/G/N/R/I/A/Q/L/S/T/E, T11E/M, R12D/N/Q/E/F, S13E, A14E/D, T16E, T61S/V, A62D, F90A/Y, Y92G/D, W155A, T157S, Q182D/E, D203C/K, N204E/G, S206D, N211D/Y/E, S212F, N213P, V219I, Y220M, Q237D, N241E/D, N243E, L247T, V170I, G135A, V167Q/T, S248C, A24R and F187I, preferably with one to twenty-five substitutions selected from F208M/W/G/N/R/I/A/Q/L/S/T/E, T11M, R12D/E/F, A14E/D, T61S/V, A62D, F90A/Y, Y92G, W155A, T157S, Q182D/E, D203C/K, N204E/G, N211D/Y/E, S212F, N213P, V219I, Q237D, N241E/D, V170I, G135A, V167Q/T, S248C, A24R and F187I, and exhibits an increased polyester degrading activity and/or an increased thermostability as compared to the amino acid sequence SEQ ID No 1 at a pH comprised between 3 and 6, preferably at a pH between 5 and 5.5.

According to the invention, the esterase may comprise at least one amino acid residue selected from S130, D175, H207, C240 or C275 as in the parent esterase of SEQ ID No 1, i.e. the enzyme of the invention is not modified at one, two, three, etc., or all of these positions.

For instance, the esterase comprises at least the amino acids S130, D175 and H207 forming the catalytic site of the enzyme and/or the amino acids C240 and C275 forming disulphide bond as in the parent esterase. Preferably, the esterase comprises at least a combination of amino acid residues selected from S130+D175+H207, C240+C275 and S130+D175+H207+C240+C275, as in the parent esterase of SEQ ID No 1.

According to the invention, the esterase disclosed in the invention exhibits a measurable polyester degrading activity at least in a range of pH from 3 to 6, from 4 to 6, from 4.5 to 6, from 5 to 6, from 5.5 to 6, from 5 to 5.5, from 5 to 5.2, from 5.2 to 5.5, from 4 to 5.5, from 4.5 to 5.5, from 5 to 5.5, preferably in a range of pH from 5 to 5.2, more preferably at pH 5.2.

The esterase may further exhibit a measurable polyester degrading activity in a pH range from 6.5 to 10, from 7 to 9.5, from 7 to 9, from 7.5 to 8.5, from 6 to 9, from 6.5 to 9, from 6.5 to 8. Preferably, the esterase further exhibits a measurable polyester degrading activity at pH 8.

Advantageously, the esterase disclosed in the invention exhibits an increased polyester degrading activity and/or an increased thermostability as compared to the esterase of SEQ ID No 1 in a range of pH between 3 and 6, preferably at a pH between 4 and 6, more preferably at a pH between 5 and 6, even more preferably at a pH between 5 and 5.5. Particularly, the esterase of the invention exhibits an increased polyester degrading activity and/or an increased thermostability as compared to the esterase of SEQ ID No 1 in the range of pH from 3 to 6, from 4 and 6, from 5 and 6, from 5 to 5.5, from 5.2 to 5.5, from 5.5 to 6, from 5 to 5.2. According to the invention, the designation of a range of pH includes the lower and upper limits of said range.

The esterase disclosed in the invention may further exhibit an increased thermostability and/or an increased polyester degrading activity as compared to the esterase of SEQ ID No 1 at a pH comprised between 6 and 10, preferably at a pH comprised between 6.5 and 9, more preferably comprised between 6.5 and 8, even more preferably at pH 8.

According to the invention, the esterase exhibits an increased polyester degrading activity and/or an increased thermostability as compared to the esterase of SEQ ID No 1 at a pH between 3 and 6. Particularly, the esterase exhibits an increased polyester degrading activity and/or an increased thermostability as compared to the esterase of SEQ ID No 1 at specific pH between 3 and 6 and/or in a range of pH between 3 and 6. Preferably, the esterase exhibits an increased polyester degrading activity and/or increased thermostability as compared to the esterase of SEQ ID No 1 at a pH comprised between 3.5 to 6, between 4 and 6, between 5 and 6, between 5 and 5.5, between 5.2 and 5.5, between 4 and 5.5, 4.5 and 5.5. Preferably, the esterase exhibits an increased polyester degrading activity and/or increased thermostability as compared to the esterase of SEQ ID No 1 at least at pH 3, at pH 3.5, at pH 4, at pH 4.5, at pH 5, at pH 5.2, at pH 5.5, and/or at pH 6. The esterase may also exhibit an increased polyester degrading activity and/or an increased thermostability as compared to the esterase of SEQ ID No 1 in the whole range of pH from 3 to 6, in the whole range of pH from 4 to 6, in the whole range of pH from 4.5 to 6, in the whole range of pH from 5 to 6, in the whole range of pH from 5.5 to 6, in the whole range of pH from 5 to 5.5, in the whole range of pH from 5 to 5.2, in the whole range of pH from 5.2 to 5.5.

According to the invention, the esterase exhibits a greater increase of polyester degrading activity and/or a greater increase of thermostability compared to the enzyme of SEQ ID No 1 at a pH comprised between 3 and 6, particularly at a pH between 5 and 5.5, than at a pH above 7, particularly greater than at a pH between 7 and 9.

In an embodiment, the esterase comprises at least one substitution and/or combination of substitution selected from N211D, N204G, F208T/M/L/S/Q/R, Q182D/E, A24R, F187I, F208M/I/T/L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L, F208M+D203C+S248C+V170I+Y92G+N213P+Q182E+D158E.

Esterases with Increased Polyester Degrading Activity

According to the invention, the esterase may comprise at least one substitution selected from N211D/Y/E, N204D/E/G, F90A/Y, F208M/W/G/N/R/I/A/Q/L/S/T/E, Q182D/E, D203C/K/R, S248C, V170I, Y92G/D, N213P/D, A24R and F187Y/I, preferably selected from N211D/E, N204G, F90A, F208M/G/N/R/I/A/Q/L/S/T, Q182D/E, D203C, S248C, V170I, Y92G, N213P A24R and F187I, more preferably selected from N211D/E, N204G, F90A, F208M/G/N/R/I/A/Q/L/S/T, Q182D/E, A24R and F187I, and exhibits an increased polyester degrading activity as compared to the enzyme of SEQ ID No 1 at a pH comprised between 3 and 6, preferably at a pH between 5 and 5.5. Advantageously, the enzyme comprises at least one substitution selected from N211D/Y/E, N204D/E/G, F208M/W/G/N/R/I/A/Q/L/S/T/E, Q182D/E, D203C/K, S248C, V170I, Y92G/D, N213P/D, A24R and F187I, preferably selected from N211D/E, N204G, F208M/R/Q/L/S/T, Q182D/E, D203C/K, S248C, V170I, Y92G, N213P A24R and F187I, more preferably selected from N211D/E, N204G, F208M/R/Q/L/S/T, Q182D/E, A24R and F187I, even more preferably selected from N211D, F208M/Q/L/S/T, Q182D/E, A24R and F187I.

Particularly, the esterase consists in the amino acid sequence set forth in SEQ ID No 1 with one one substitution selected from N211D/Y/E, N204D/E/G, F208M/W/G/N/R/I/A/Q/L/S/T/E, Q182D/E, D203C/K, S248C, V170I, Y92G/D, N213P/D, A24R and F187I, preferably selected from N211D/E, N204G, F208M/R/Q/L/S/T, Q182D/E, D203C/K, S248C, V170I, Y92G, N213P A24R and F187I, more preferably selected from N211D/E, N204G, F208M/R/Q/L/S/T, Q182D/E, A24R and F187I, even more preferably selected from N211D, F208M/Q/L/S/T, Q182D/E, A24R and F187I.

According to the invention, the enzyme may comprise at least the combination of substitution selected from F208M+D203C+S248C+V170I+Y92G+N213P+Q182E, F208I+D203C+S248C+V170I+Y92G+N213P+Q182E and F208M+D203C+S248C+V170I+Y92G+N213P+Q182E+S13L+D158E.

Indeed, the inventors have shown that these esterases exhibit a greater increase of polyester degrading activity compared to the enzyme of SEQ ID No 1 in acidic conditions than in basic conditions. Particularly, the inventors have shown that said esterases are more efficient, comparatively to the parent esterase (i.e. exhibit a greater increase of polyester degrading activity compared to the enzyme of SEQ ID No 1), at a pH between 3 and 6, particularly at a pH between 5 and 5.5, than at a pH above 7, particularly at a pH between 7 and 9.

For instance, the esterase comprises at least one combination of substitutions selected from F208M/W/G/N/R/I/A/Q/L/S/T/E+D203C+S248C+V170I+Y92G/D+N213P/D+Q182D/E and F208M/W/G/N/R/I/A/Q/L/S/T/E+D203K+V170I+Y92G/D+N213P/D+Q182D/E, preferably selected from F208M/I/L/T+D203C+S248C+V170I+Y92G+N213P+Q182E and F208M/I/L/T+D203K+V170I+Y92G/D+N213P/D+Q182D/E, more preferably selected from F208M/I/L/T+D203C+S248C+V170I+Y92G+N213P+Q182E. Particularly, the enzyme may comprise at least a combination of substitutions selected from F208M/I+D203C+S248C+V170I+Y92G+N213P+Q182E. Particularly, the enzyme may comprise at least a combination of substitutions selected from F208M/I+D203C+S248C+V170I+Y92G+N213P+Q182E+S13L+D158E.

In a particular embodiment, the esterase consists in the amino acid sequence set forth in SEQ ID No 1 with one combination of substitutions selected from F208M+D203 C+S248C+V170I+Y92G+N213P+Q182E, F208I+D203C+S248C+V170I+Y92G+N213P+Q182E and F208M+D203C+S248C+V170I+Y92G+N213P+Q182E+S13L+D158E.

In an embodiment, the esterase comprises the amino acid sequence set forth in SEQ ID No 1 with one to twelve substitutions selected from N211D/Y/E, N204D/E/G, F90A/Y, F208M/W/G/N/R/I/A/Q/L/S/T/E, Q182D/E, D203C/K, S248C, V170I, Y92G/D, N213P/D, A24R and F187Y/I, preferably selected from N211D/E, N204G, F90A, F208M/G/N/R/I/A/Q/L/S/T, Q182D/E, D203C, S248C, V170I, Y92G, N213P, A24R and F187I, and exhibits an increased polyester degrading activity as compared to the amino acid sequence SEQ ID No 1 at a pH comprised between 3 and 6, preferably at a pH between 5 and 5.5. Particularly, the esterase comprises the amino acid sequence set forth in SEQ ID No 1 with one to eleven substitutions selected from N211D/Y/E, N204D/E/G, F208M/W/G/N/R/I/A/Q/L/S/T/E, Q182D/E, D203C/K, S248C, V170I, Y92G/D, N213P/D, A24R and F187Y/I, preferably selected from N211D, N204G, F208NMR/IQ/L/S/T, Q182D/E, D203C/K, S248C, V170I, Y92G, N213P, A24R and F187I, more preferably one to ten substitutions selected from N211D/E, F208M/I/Q/L/S/T, Q182E, D203C/K, S248C, V170I, Y92G, N213P, A24R and F187I and exhibits a greater increase of polyester degrading activity compared to the esterase of SEQ ID No 1 in acidic conditions than in basic conditions.

In an embodiment, the esterase consists in the amino acid sequence set forth in SEQ ID No 1 with one to twelve substitutions selected from N211D/Y/E, N204D/E/G, F90A/Y, F208M/W/G/N/R/I/A/Q/L/S/T/E, Q182D/E, D203C/K, S248C, V170I, Y92G/D, N213P/D, A24R and F187Y/I, preferably selected from N211D/E, N204G, F90A, F208M/G/N/R/I/A/Q/L/S/T, Q182D/E, D203C, S248C, V170I, Y92G, N213P, A24R and F187I, and exhibits an increased polyester degrading activity as compared to the amino acid sequence SEQ ID No 1 at a pH comprised between 3 and 6, preferably at a pH between 5 and 5.5. Particularly, the esterase consists in the amino acid sequence set forth in SEQ ID No 1 with one to eleven substitutions selected from N211D/Y/E, N204D/E/G, F208M/W/G/N/R/I/A/Q/L/S/T/E, Q182D/E, D203C/K, S248C, V170I, Y92G/D, N213P/D, A24R and F187Y/I, preferably selected from N211D, N204G, F208NMR/I/Q/L/S/T, Q182D/E, D203C/K, S248C, V170I, Y92G, N213P, A24R and F187I, more preferably one to ten substitutions selected from N211D/E, F208M/I/Q/L/S/T, Q182E, D203C/K, S248C, V170I, Y92G, N213P, A24R and F187I and exhibits a greater increase of polyester degrading activity compared to the esterase of SEQ ID No 1 in acidic conditions than in basic conditions.

In an embodiment, the esterase has the amino acid sequence set forth in SEQ ID No 1 with a single substitution selected from N211D/Y/E, N204D/E/G, F90A/Y, F208M/W/G/N/R/I/A/Q/L/S/T/E, Q182D/E, A24R and F187Y/I, preferably selected from N211D/E, N204G, F90A, F208M/G/N/R/I/A/Q/L/S/T, Q182D/E, A24R and F187I, and exhibits an increased polyester degrading activity as compared to the amino acid sequence SEQ ID No 1 at a pH comprised between 3 and 6, preferably at a pH between 5 and 5.5. Particularly, the esterase has the amino acid sequence set forth in SEQ ID No 1 with a single substitution selected from N211D/E, N204G, F208M/R/Q/L/S/T, Q182E, A24R and F187I preferably selected from F208T/L/M/S/R and Q182E. Advantageously, said esterase exhibits a greater increase of polyester degrading activity compared to the enzyme of SEQ ID No 1 in acidic conditions than in basic conditions.

Esterases with Increased Thermostability

According to the invention, the esterase may comprise at least one substitution selected from N211D/Y/E, N204D/E/G, F208M/W/G/N/R/I/A/Q/L/S/T/E, D203C/K/R, S248C, V170I, Y92G/D, N213P/D and Q182D/E, preferably selected from N211D, N240G, F208M/I/L/T, D203C, S248C, V170I, Y92G, N213P and Q182E, more preferably selected from N211D, N240G, and exhibits an increased thermostability as compared to the esterase of SEQ ID No 1 at a pH comprised between 3 and 6, preferably at a pH between 5 and 5.5.

Advantageously, the esterase comprises at least one combination of substitutions selected from F208M/W/G/N/R/I/A/Q/L/S/T/E+D203C+S248C+V170I+Y92G/D+N213P/D+Q182D/E and F208M/W/G/N/R/I/A/Q/L/S/T/E+D203K+V170I+Y92G/D+N213P/D+Q182D/E, preferably selected from F208M/I/L/T+D203C+S248C+V170I+Y92G+N213P+Q182E and F208M/I/L/T+D203K+V170I+Y92G/D+N213P/D+Q182D/E, more preferably selected from F208M/I/L/T+D203C+S248C+V170I+Y92G+N213P+Q182E.

Advantageously, the esterase comprises at least one combination of substitutions selected from F208I+D203C+S248C+V170I+Y92G+N213P+Q182E, F208M+D203C+S248C+V170I+Y92G+N213P+Q182E, F208T+D203C+S248C+V170I+Y92G+N213P+Q182E, F208L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L and F208M+D203C+S248C+V170I+Y92G+N213P+Q182E+D158E.

Indeed, the inventors have shown that these esterases exhibit a greater increase of thermostability, compared to the enzyme of SEQ ID No 1, in acidic conditions than in basic conditions. Particularly, the inventors have shown that said esterases are more thermostable, comparatively to the parent esterase, at a pH between 3 and 6, particularly at a pH between 5 and 5.5, than at a pH above 7, particularly at a pH between 7 and 9.

In a particular embodiment, the esterase consists in the amino acid sequence set forth in SEQ ID No 1 with one combination of substitutions selected from F208I+D203C+S248C+V170I+Y92G+N213P+Q182E, F208M+D203C+S248C+V170I+Y92G+N213P+Q182E, F208T+D203C+S248C+V170I+Y92G+N213P+Q182E, F208L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L and F208M+D203C+S248C+V170I+Y92G+N213P+Q182E+D158E.

Esterases with Both Increased Degrading Activity and Increased Thermostability

According to the invention, the esterase may comprise at least one substitution selected from N211D/Y/E, N204D/E/G, F208M/W/G/N/R/I/A/Q/L/S/T/E, D203C/K/R, S248C, V170I, Y92G/D, N213P/D and Q182D/E, preferably selected from N211D, N204G, F208M/I/L/T, D203C/K, S248C, V170I, Y92G, N213P and Q182E, more preferably selected from N211D and N204G, and exhibits both an increased polyester degrading activity and an increased thermostability as compared to the amino acid sequence SEQ ID No 1 at a pH comprised between 3 and 6, preferably at a pH between 5 and 5.5.

For instance, the esterase comprises at least one combination of substitutions selected from F208M/W/G/N/R/I/A/Q/L/S/T/E+D203C+S248C+V170I+Y92G/D+N213P/D+Q182D/E and F208M/W/G/N/R/I/A/Q/L/S/T/E+D203K+S248C+V170I+Y92G/D+N213P/D+Q182D/E, preferably selected F208M/I/L/T+D203C+S248C+V170I+Y92G/D+N213P/D+Q182D/E and F208M/I/L/T+D203K+S248C+V170I+Y92G/D+N213P/D+Q182D/E, more preferably the combination of substitutions F208M/I+D203C+S248C+V170I+Y92G+N213P+Q182E.

Indeed, the inventors have shown that these esterases exhibit a greater increase of polyester degrading activity and a greater increase of thermostability, compared to the enzyme of SEQ ID No 1 in acidic conditions than in basic conditions. Particularly, the inventors have shown that said esterases are more efficient and stable, comparatively to the parent esterase, at a pH between 3 and 6 than at a pH above 7, particularly at a pH between 7 and 9.

In another embodiment, the esterase has the amino acid sequence set forth in SEQ ID No 1 with one to nine substitutions selected from N211D/Y/E, N204D/E/G, F208M/W/G/N/R/I/A/Q/L/S/T/E, D203C/K, S248C, V170I, Y92G/D, N213P/D and Q182D/E, preferably selected from N211D, N204G, F208M/I/L/T, D203C/K, S248C, V170I, Y92G, N213P and Q182E, and exhibits both an increased polyester degrading activity and an increased thermostability as compared to the amino acid sequence SEQ ID No 1 at a pH comprised between 3 and 6, preferably at a pH between 5 and 5.5. As an example, the esterase has the amino acid sequence set forth in SEQ ID No 1 with a single substitution selected from N204D/E/G and N211D/Y/E, preferably selected from N204G and N211D.

In another example, the esterase has the amino acid sequence set forth in SEQ ID No 1 with one combination of substitutions selected from F208M/W/G/N/R/I/A/Q/L/S/T/E+D203C+S248C+V170I+Y92G/D+N213P/D+Q182D/E, F208M/W/G/N/R/I/A/Q/L/S/T/E+D203K+S248C+V170I+Y92G/D+N213P/D+Q182D/E, F208M/W/G/N/R/I/A/Q/L/S/T/E+D203C+S248C+V170I+Y92G+N213P+Q182E+S13L+D158E and F208M/W/G/N/R/I/A/Q/L/S/T/E+D203K+V170T+Y92G+N213P+Q182E+S13L+D158E, preferably selected F208M/I/L/T+D203C+S248C+V170I+Y92G/D+N213P/D+Q182D/E, F208M/I/L/T+D203K+S248C+V170I+Y92G/D+N213P/D+Q182D/E, F208M/I/L/T+D203C+S248C+V170I+Y92G+N213P+Q182E+S13L+D158E and F208M/I/L/T+D203K+V170I+Y92G+N213P+Q182E+S13L+D158E, more preferably F208M/I+D203C+S248C+V170I+Y92G+N213P+Q182E or F208M/I+D203C+S248C+V170I+Y92G+N213P+Q182E+S13L+D158E. Particularly, said esterase exhibits a greater increase of polyester degrading activity and a greater increase of thermostability, compared to the enzyme of SEQ ID No 1 in acidic conditions than in basic conditions. Particularly, the esterase has the amino acid sequence set forth in SEQ ID No 1 with the combination of substitutions F208M/I+D203C+S248C+V170I+Y92G+N213P+Q182E or and F208M+D203C+S248C+V170I+Y92G+N213P+Q182E+S13L+D158E, and is more efficient and stable, comparatively to the parent esterase, at a pH between 3 and 6, than at a pH above 7, particularly at a pH between 7 and 9.

### Degrading Process

By working on the optimisation of enzymatic degrading process of polyester or polyester containing material, the inventors have discovered that it is possible to avoid coproducts (salts) production and to improve the economic return of a polyester or polyester containing material degrading process by reducing the base consumption, while maintaining an enzymatic activity compatible with industrial performances. More particularly, the inventors have discovered that an enzymatic depolymerization of polyester may be performed at an acid pH, without addition of any base, when the reaction medium already contains a certain amount of equivalent terephthalic acid in the form of salts. The specific esterases described above are particularly suited for degrading a polyester or polyester containing material in acidic conditions.

In this regard, it is an object of the invention to provide a process for degrading a polyester or a polyester containing material, wherein the process comprises a step of depolymerization of said polyester performed by contacting the polyester containing material or the polyester in a reaction medium at a pH between 3 and 6, with any esterase as described above, able to degrade said polyester.

According to the invention, the depolymerization step is preferably implemented at a temperature between 40° C. and 80° C., preferably between 50° C. and 72° C., more preferably between 50° C. and 65° C., even more preferably between 50° C. and 60° C. In an embodiment, the depolymerization step is implemented at a temperature between 55° C. and 60° C. or between 50° C. and 55° C. In another embodiment, the depolymerization step is implemented between 55° C. and 65° C. In another embodiment, the depolymerization step is implemented between 60° C. and 72° C., preferably between 60° C. and 70° C. Advantageously, the temperature of the depolymerization step is maintained below the Tg of the polyester of interest. Within the context of the invention, the “polyester of interest” refers to the polyester targeted by the depolymerization step. Preferably, the polyester of interest comprises at least a terephthalic acid monomer (TA). Advantageously, the temperature is maintained at a given temperature+/−1° C.

According to the invention, the depolymerization step may be implemented at a pH between 4 and 6, between 5 and 6, between 4 and 5.5, between 4.5 and 5.5, between 5 and 5.5, between 5.5 and 6, between 5.1 and 5.3, between 5 and 5.2, between 3 and 5, between 3 and 4, between 3.5 and 4, between 4 and 5 or between 4.5 and 5.

Advantageously, the depolymerization step is implemented at a pH between 5.0 and 5.5, such as at pH 5.2, and at a temperature comprised between 50° C. and 72° C., preferably between 50° C. and 65° C. or between 65° C. and 72° C.

### Additional Enzymes

The process for degrading a polyester or a polyester containing material may be implemented by contacting the polyester or the polyester containing material with any one of the esterase disclosed above and at least one additional enzyme.

For instance, the additional enzyme is a cutinase coming from a microorganism selected from Thermobifida cellulosityca, Thermobifida halotolerans, Thermobifida fusca, Thermobifida alba, Bacillus subtilis, Fusarium solani pisi, Humicola insolens, Sirococcus conigenus, Pseudomonas mendocina, Thielavia terrestris, Saccharomonospora viridis, Thermomonospora curvata or any functional variant thereof having a cutinase activity Alternatively or in addition, the additional enzyme may be a lipase, preferably coming from Ideonella sakaiensis and/or a cutinase coming from Humicola insolens, such as the one referenced A0A075B5G4 in Uniprot or any functional variant thereof having a cutinase activity. The additional enzyme may also be selected from commercial enzymes such as Novozym 51032 or any functional variant thereof.

Alternatively or in addition, the process for degrading a polyester or a polyester containing material may be implemented by contacting the polyester with any one of the esterases disclosed above and with an enzyme having a MHET-degrading activity (MHETase).

The quantity of esterase(s) in the reaction medium may be comprised between 0.1 mg and 15 mg of esterase/g of the targeted polyester, preferably comprised between 0.1 mg/g and 10 mg/g, more preferably comprised between 0.1 mg/g and 5 mg/g, even more preferably between 0.5 mg/g and 4 mg/g. Preferably, the quantity of esterase(s) in the reaction medium is at most of 4 mg/g of the targeted polyester, preferably at most of 3 mg/g, more preferably at most 2 mg/g of the targeted polyester.

More generally, the quantity of each enzyme in the reaction medium is preferably comprised between 0.1 mg and 15 mg/g of the targeted polymer(s).

### Polyesters

The claimed invention may be implemented with any polyester or polyester containing material. Preferably, the polyester is selected from polyethylene terephthalate (PET), polytrimethylene terephthalate (PTT), polybutylene terephthalate (PBT), polyethylene isosorbide terephthalate (PEIT), polybutylene adipate terephthalate (PBAT), polycyclohexylenedimethylene terephthalate (PCT), glycosylated polyethylene terephthalate (PETG), poly (butylene succinate-co-terephtalate) (PBST), poly(butylene succinate/terephthalate/isophthalate)-co-(lactate) (PBSTIL), “polyolefin-like” polyesters and blends/mixtures of these polymers. Preferably, the polyester is polyethylene terephthalate (PET).

For instance, the invention may be implemented with plastic products from plastic waste collection and/or post-industrial waste. More particularly, the invention may be implemented for degrading domestic plastic wastes, including plastic bottles, plastic trays, plastic bags, plastic packaging, soft plastics and/or hard plastics, even polluted with food residues, surfactants, etc. Alternatively, or in addition, the invention may be implemented for degrading plastic fibers, such as fibers providing from fabrics, textiles and/or industrial wastes. More particularly, the invention may be used with PET plastic and/or PET fiber waste, such as PET fibers coming from fabrics, textile, and/or tires.

The invention may be particularly adapted for degrading plastic products comprising at least one polyester comprising at least a terephthalic acid monomer (TA).

### Purification

The process for degrading plastic product may further comprise a step of recovering and optionally purifying the monomers and/or oligomers and/or degradation products, preferably terephthalic acid, resulting from the step(s) of depolymerization. Monomers and/or oligomers and/or degradation products resulting from the depolymerization may be recovered, sequentially or continuously.

A single type of monomers and/or oligomers or several different types of monomers and/or oligomers may be recovered. The recovered monomers and/or oligomers and/or degradation products may be purified, using all suitable purifying method and optionally conditioned in a re-polymerizable form. An example of purification is described in the patent application WO 1999/023055. For instance, the recovery of TA under solid form comprises separating the solid phase from the liquid phase of the reaction medium by filtration.

The solid phase recovered may be dissolved in a solvent selected from DMF, NMP, DMSO, DMAC or any solvent known to solubilized TA and filtered to remove impurities. Solubilized TA can then be recrystallized by any means known by one skilled in the art.

After the depolymerization step, a MHETase may be added in the reaction medium before the purification process, in order to hydrolyze the MHET produced during the depolymerization step(s) to produce TA.

The repolymerizable monomers and/or oligomers may then be reused to synthesize polymers. One skilled in the art may easily adapt the process parameters to the monomers/oligomers and the polymers to synthesize.

Accordingly, it is also an object of the invention to provide a process for recycling a polyester or a polyester of a polyester containing material, by contacting said polyester or polyester containing material at a pH between 3 and 6 with any one of the esterases described above, and/or to provide a method of producing monomers and/or oligomers and/or degradation products, from a polyester containing material, comprising submitting the polyester containing material to a depolymerization step performed at a pH between 3 and 6 by using any one of the esterases described above, and recovering and optionally purifying the monomers and/or oligomers.

All embodiments exposed above in connection with the process for degrading plastic product also apply to the methods of producing monomers and/or oligomers and to the methods of recycling.

### Other Uses of Esterases

The invention also relates to the use of any one of the esterases described above in a method of surface hydrolysis or surface functionalization of a polyester containing material, comprising exposing a polyester containing material to said esterase at a pH between 3 and 6. Said method is particularly useful for increasing hydrophilicity, or water absorbency, of a polyester material. Such increased hydrophilicity may have particular interest in textiles production, electronics and biomedical applications.

The invention also relates to the use of any one of the esterases described above for treating water, waste water or sewage at a pH between 3 and 6. In waste water or sewage treatment applications said esterase can be used to degrade microplastic particles consisting of polyester (preferable PET) like polymer filaments, fibres or other kinds of polyester-based product debris and fragments, preferably PET-based product debris and fragments.

The invention also relates to the use of any one of the esterases described above in detergent, food, animal feed, paper making, textile and pharmaceutical applications. As an example, any one of the esterases described above can be used at a pH between 3 and 6 in papermaking industry. More particularly, said esterases may be used to remove stickies from the paper pulp and water pipelines of paper machines.

### EXAMPLES

### Example 1—Construction, Expression and Purification of Esterases

### Construction

Esterase according to the invention have been generated using the plasmidic construction pET26b-LCC-His. This plasmid consists in cloning a gene encoding the esterase of SEQ ID No 1, optimized for Escherichia coli expression between NdeI and XhoI restriction sites. Two site directed mutagenesis kits have been used according to the recommendations of the supplier, in order to generate the esterase variants: QuikChange II Site-Directed Mutagenesis kit and QuikChange Lightning Multi Site-Directed from Agilent (Santa Clara, California, USA).

### Expression and Purification of the Esterases

The strains Stellar™ (Clontech, California, USA) and E. coli BL21 (DE3) (New England Biolabs, Evry, France) have been successively employed to perform the cloning and recombinant expression in 50 mL LB-Miller medium or ZYM auto inducible medium (Studier et al., 2005—Prot. Exp. Pur. 41, 207-234). The induction in LB-Miller medium has been performed at 16° C., with 0.5 mM of isopropyl P-D-1-thiogalactopyranoside (IPTG, Euromedex, Souffelweyersheim, France). The cultures have been stopped by centrifugation (8000 rpm, 20 minutes at 10° C.) in an Avanti J-26 XP centrifuge (Beckman Coulter, Brea, USA). The cells have been suspended in 20 mL of Talon buffer (Tris-HCl 20 mM, NaCl 300 mM, pH 8). Cell suspension was then sonicated during 2 minutes with 30% of amplitude (2 sec ON and 1 sec OFF cycles) by FB 705 sonicator (Fisherbrand, Illkirch, France). Then, a step of centrifugation has been realized: 30 minutes at 10000 g, 10° C. in an Eppendorf centrifuge. The soluble fraction has been collected and submitted to affinity chromatography. This purification step has been completed with Talon® Metal Affinity Resin (Clontech, CA, USA). Protein elution has been carried out with steps of Talon buffer supplemented with imidazole. Purified protein has been dialyzed against Talon buffer or sodium acetate buffer (100 to 300 mM, pH 5.2) then quantified using Bio-Rad protein assay according to manufacturer instructions (Lifescience Bio-Rad, France) and stored at +4° C.

### Example 2—Evaluation of the Degrading Activity of the Esterases

The degrading activity of the esterases has been determined and compared to the activity of esterase of SEQ ID No 1.

Multiple methodologies to assess the specific activity have been used:
(1) Specific activity based upon PET hydrolysis and Ultra High-Performance Liquid Chromatography (UHPLC) analysis
(2) Specific activity based upon PET hydrolysis and Ultraviolet Light Absorbance (UV Assay) analysis
(3) Degrading activity based upon the degradation of a polyester under solid form
(4) Degrading activity based upon PET hydrolysis in reactors above 100 mL

### 2.1. Specific Activity Based Upon PET Hydrolysis and Ultra High-Performance Liquid Chromatography (UHPLC) Analysis

100 mg of amorphous PET under powder form (prepared according to WO 2017/198786 to reach a crystallinity below 20%) were weighted and introduced in a 100 mL glass bottle. 1 mL of esterase preparation comprising esterase of SEQ ID No 1 (as reference control) or esterase of the invention, prepared at 1.727 μM in sodium acetate buffer (100 to 300 mM, pH 5.2) for measure in acidic conditions (or at 0.69 μM in Talon buffer (Tris-HCl 20 mM, NaCl 0.3M, pH 8) in basic conditions) were introduced in the glass bottle. Finally, 9 mL or 49 mL of the corresponding buffer (according to the pH to which the measure will be made) were added.

The depolymerization started by incubating each glass bottle at 50° C., 54° C., 60° C., 65° C., 68° C. or 72° C. and 150 rpm in a Max Q 4450 incubator (Thermo Fisher Scientific, Inc. Waltham, MA, USA).

The initial rate of depolymerization reaction, in mg of equivalent TA generated/hour, was determined by samplings performed at different time during the first 24 hours and analyzed by Ultra High Performance Liquid Chromatography (UHPLC). If necessary, samples were diluted in 0.1 M potassium phosphate buffer pH 8. Then, 150 μL of methanol and 6.5 μL of HCl 6 N were added to 150 μL of sample or dilution. After mixing and filtering on 0.45 μm syringe filter, samples were loaded on UHPLC to monitor the liberation of terephthalic acid (TA), MHET and BHET. Chromatography system used was an Ultimate 3000 UHPLC system (Thermo Fisher Scientific, Inc. Waltham, MA, USA) including a pump module, an autosampler, a column oven thermostated at 25° C., and an UV detector at 240 nm. The column used was a Discovery® HS C18 HPLC Column (150×4.6 mm, 5 μm, equipped with precolumn, Supelco, Bellefonte, USA). TA, MHET and BHET were separated using a gradient of MeOH (30% to 90%) in 1 mM of H₂SO₄ at 1 mL/min. Injection was 20 μL of sample. TA, MHET and BHET were measured according to standard curves prepared from commercial TA and BHET and in house synthetized MHET in the same conditions than samples. The specific activity of PET hydrolysis (mg of equivalent TA/hour/mg of enzyme) was determined in the linear part of the hydrolysis curve of the reaction (i.e. at the beginning of the reaction), such curve being set up by samplings performed at different time during the first 24, 48 or 72 hours. Equivalent TA corresponds to the sum of TA measured and of TA contained in measured MHET and BHET. Said measurement of equivalent TA can also be used to calculate the yield of a PET depolymerization assay at a given time and/or after a defined period of time (e.g. 24h or 48h).

### 2.2 Specific Activity Based Upon PET Hydrolysis and Ultraviolet Light Absorbance (UV Assay) Analysis

100 mg of amorphous PET under powder form (prepared according to WO 2017/198786 to reach a crystallinity below 20%) were weighted and introduced in a 100 mL glass bottle. 1 mL of esterase preparation comprising esterase of SEQ ID No 1 (as reference control) or esterase of the invention, prepared at 1,727 μM in sodium acetate buffer (100 to 300 mM, pH 5.2) for measure in acidic conditions (or at 0.69 μM in Talon buffer (Tris-HCl 20 mM, NaCl 0.3M, pH 8) in basic conditions) were introduced in the glass bottle. Finally, 9 mL or 49 mL of the corresponding buffer (according to the pH to which the measure will be made) were added.

The depolymerization started by incubating each glass bottle at 50° C., 54° C., 60° C. or 65° C. and 150 rpm in a Max Q 4450 incubator (Thermo Fisher Scientific, Inc. Waltham, MA, USA).

The initial rate of depolymerization reaction, in μmol of soluble degradation products generated/hour was determined by samplings performed at different time during the first 24 hours and analyzed by absorbance reading at 242 nm using an Eon Microplate Spectrophotometer (BioTek, USA). The increase in absorbance of the reaction mixtures in the ultraviolet region of the light spectrum (at 242 nm) indicates the release of soluble TA or its esters (BHET and MHET) from an insoluble PET substrate. The absorbance value at this wavelength can be used to calculate the overall sum of PET hydrolysis products according to the Lambert-Beer law, and the enzyme-specific activity is determined as total equivalent TA produced. The specific activity of PET hydrolysis (μmol of soluble products/hour/mg of enzyme) was determined in the linear part of the hydrolysis curve of the reaction (i.e. at the beginning of the reaction), such curve being set up by samplings performed at different time during the first 24, 48 or 72 hours. Said measurement of equivalent TA can also be used to calculate the yield of a PET depolymerization assay at a given time and/or after a defined period of time (e.g. 24h or 48h). If necessary, samples were diluted in 0.1 M potassium phosphate buffer pH 8.

### 2.3. Activity Based Upon Degradation of a Polyester Under Solid Form

Preparation of agar plates was realized by solubilizing 50 mg of PET in hexafluoro-2-propanol (HIP) and pouring this medium in a 250 mL aqueous solution. After HFIP evaporation at 50° C. under 140 mbar, the solution was mixed with potassium phosphate buffer pH 8.0 or with sodium acetate buffer pH 5.2 or with sodium acetate buffer pH 5.0 to obtain a final concentration of 0.5 mg/mL of PET and 0.1 M of buffer containing 1% agar. Around 30 mL of the mixture is used to prepare each plate and stored at 4° C. 1 μL, 5 μL or 20 μL of enzyme preparation (pure enzyme or cell lysate) was deposited in a well created in an agar plate containing PET at pH 8.0, 5.2 or 5.0, respectively.

The diameters or the surface area of the halos formed due to the polyester degradation by wild-type esterase and variants were determined by measuring the diameter of the halos on agar plates pictures using the software Gimp and compared after a defined period of time (from 2 to 24 hours) at 40° C., 45° C., 50° C., 55° C., 60° C., 65° C. or 70° C.

### 2.4. Activity Based Upon PET Hydrolysis in Reactor

From 0.69 μmol to 2.07 μmol of purified esterase prepared in 80 mL of 100 mM potassium phosphate buffer pH 8 or 300 mM sodium acetate buffer pH 5.0, or 300 mM sodium acetate buffer pH 5.2, or 300 mM sodium acetate buffer pH 6.0 were mixed with 20 g amorphous PET (prepared according to WO 2017/198786 to reach a crystallinity below 20%) in a 500 mL Minibio bioreactor (Applikon Biotechnology, Delft, The Netherlands). Temperature regulation at 40° C., 45° C., 50° C., 55° C., 60° C., 65° C. or 70° C. was performed by water bath immersion and a single marine impeller was used to maintain constant agitation at 250 rpm. The pH of the PET depolymerization assay was regulated at pH 5 or pH 5.2 or pH 6 or pH 8 by addition of 6N NaOH and was assured by my-Control bio controller system (Applikon Biotechnology, Delft, The Netherlands). Base consumption was recorded during the assay and may be used for the characterization of the PET depolymerization assay.

The final yield of the PET depolymerization assay was determined either by the determination of residual PET weight or by the determination of equivalent TA generated, or through the base consumption. Weight determination of residual PET was assessed by the filtration, at the end of the reaction, of the reactional volume through a 12 to 15 μm grade 11 ashless paper filter (Dutscher SAS, Brumath, France) and drying of such retentate before weighting it. The determination of equivalent TA generated was realized using UHPLC methods described in 2.1, and the percentage of hydrolysis was calculated based on the ratio of molar concentration at a given time (TA+MHET+BHET) versus the total amount of TA contained in the initial sample. PET depolymerization produced acid monomers that will be neutralized with the base to be able to maintain the pH in the reactor. The determination of equivalent TA produced was calculating using the corresponding molar base consumption, and the percentage of hydrolysis was calculated based on the ratio of molar concentration at a given time of equivalent TA versus the total amount of TA contained in the initial sample.

### Results

### Activity Based Upon Degradation of PET Under Solid Form Under Acidic Condition as Compared to the Esterase of SEQ ID No 1

The activity of the esterases (variants) of the invention was evaluated after 24 hours at 50° C. and at pH 5.0 (V1, V3-V5, V9, V29-V35, V46-48) or pH 5.2 (V2, V20-V28, V36-42, V44-V45, V49-V68) as exposed in Example 2.3. The surface area of the halos of the esterases (variants) of the invention was compared to the surface area formed by the wild-type esterase of SEQ ID No 1. Variants having a greater surface area than the wild-type esterase of SEQ ID No 1 (i.e. having a better degrading activity than esterase of SEQ ID No 1 after the defined period of time) are reported in Table 1 below.

TABLE 1
Variants having an increased activity as compared
to esterase of SEQ ID No 1, based upon degradation of
a polyester under solid form after 24 hours at 50° C.

| Variants of the invention | Variants of the invention |
|---|---|
| V20: T11E | V44: S206D |
| V21: R12D | V45: S206E |
| V22: R12N | V46: F208W |
| V23: R12Q | V9: F208I |
| V24: S13E | V5: F208L |
| V25: A14E | V4: F208T |
| V26: A14D | V47: F208E |
| V27: T16E | V1: N211D |
| V28: A17T | V48: N211Y |
| V29: T61S | V49: S212F |
| V30: T61V | V50: N213P |
| V3: F90A | V51: N213D |
| V31: F90Y | V52: N214D |
| V32: Y92G | V53: A215N |
| V33: Y92D | V54: S218A |
| V34: W155A | V55: Y220M |
| V35: T157S | V56: Y220F |
| V36: P179D | V57: Q237D |
| V37: P179E | V58: F238E |
| V38: Q182D | V59: N241E |
| V39: Q182E | V60: N241D |
| V40: F187Y | V61: N243E |
| V41: N204D | V62: N243D |
| V42: N204E | V63: L247T |
| V2: N204G | V43: A205D |
| V64: T11M | V65: R12E |
| V66: R12F | V67: A62D |
| V68: V219I | |

Variants listed in Table 1 have the exact amino acid sequence as set forth in SEQ ID No 1, except the substitutions listed in Table 1, respectively.

Interestingly, most of the variants show halos having a diameter equal to or greater than 110% of the halo diameter of the wild-type esterase of SEQ TD No 1. These variants are reported in Table 2 below.

TABLE 2
Variants forming a halo diameter equal to or greater than 110% of the
halo diameter formed by the esterase of SEQ ID No 1, based upon
degradation of a polyester under solid form after 24 hours at 50° C.

| Variants of | Diameter of halo |
|---|---|
| the invention | compared to SEQ ID No 1 |
| V1: N211D | 158% |
| V3: F90A | 163% |
| V4: F208T | 133% |
| V5: F208L | 158% |
| V9: F208I | 224% |
| V21: R12D | 119% |
| V25: A14E | 112% |
| V26: A14D | 112% |
| V29: T61S | 173% |
| V30: T61V | 127% |
| V31: F90Y | 167% |
| V34: W155A | 157% |
| V35: T157S | 111% |
| V38: Q182D | 115% |
| V39: Q182E | 116% |
| V42: N204E | 110% |
| V46: F208W | 127% |
| V47: F208E | 167% |
| V48: N211Y | 200% |
| V49: S212F | 126% |
| V50: N213P | 131% |
| V57: Q237D | 112% |
| V59: N241E | 111% |
| V60: N241D | 116% |
| V64: T11M | 110% |
| V65: R12E | 122% |
| V66: R12F | 121% |
| V67: A62D | 119% |
| V68: V219I | 122% |

Variants listed in Table 2 have the exact amino acid sequence as set forth in SEQ ID No 1, except substitutions listed in Table 2, respectively.

### Specific Degrading Activity Under Acidic Condition as Compared to the Esterase of SEQ ID No 1

Specific degrading activity of esterases (variants) used according to the invention has been determined from the linear part of the hydrolysis curve, i.e. at the beginning of the reaction. The specific degrading activity of the esterase of SEQ TD No 1 is used as a reference and considered as 100% specific degrading activity. The specific degrading activity was measured at pH 5.2 as exposed in Example 2.1 at 54° C. for variants V1 to V15 and at 60° C. for variants V16 to V19 and V69 to V72. The results are summarized in Table 3 below.

TABLE 3
Specific degrading activity of variants used in the
invention at pH 5.2, compared to SEQ ID No 1.

| | Specific |
|---|---|
| | degrading activity |
| | compared to SEQ ID |
| Variants | No 1 at pH 5.2 (%) |
| V1: N211D | 384% |
| V2: N204G | 112% |
| V3: F90A | 119% |
| V4: F208T | 182% |
| V5: F208L | 175% |
| V6: F208M | 165% |
| V7: F208S | 155% |
| V8: F208Q | 144% |
| V9: F208I | 140% |
| V10: F208A | 139% |
| V11: F208R | 138% |
| V12: F208N | 118% |
| V13: F208G | 113% |
| V14: Q182E | 149% |
| V15: Q182D | 130% |
| V16: F208M + D203C + S248C + | 445% |
| V170I + Y92G + N213P + Q182E | |
| V17: F208T + D203C + S248C + | 500% |
| V170I + Y92G + N213P + Q182E | |
| V18: F208L + D203C + S248C + | 526% |
| V170I + Y92G + N213P + Q182E | |
| V19: F208I + D203C + S248C + | 468% |
| V170I + Y92G + N213P + Q182E | |
| V69: N211E | 140% |
| V70: F208M + D203C + S248C + | 723% |
| V170I + Y92G + N213P + | |
| Q182E + S13L+ D158E | |
| V71: F208M + D203C + S248C + | 539% |
| V170I + Y92G + N213P + | |
| Q182E + S13L | |
| V72: F208M + D203C + S248C + | 637% |
| V170I + Y92G + N213P + | |
| Q182E + D158E | |

Variants of Table 3 have the exact amino acid sequence as set forth in SEQ ID No 1, except the substitution or combination of substitutions listed in Table 3, respectively.

### PET Depolymerization Yield Under Acidic Condition as Compared to the Esterase of SEQ ID No 1

The PET depolymerization yield of esterases (variants) used according to the invention, after 24 hours at pH 5.2 and 54° C., are shown in Table 4 below. The PET depolymerization yield of the esterase of SEQ ID No 1 is used as a reference and considered as 100% PET depolymerization yield. The PET depolymerization yield was measured as exposed in Example 2.1 after 24 hours for variant V73 and as exposed in Example 2.2 for variant V74.

TABLE 4
PET depolymerization yield of the esterase of the invention
after 24 hours at pH 5.2 and 54° C. compared to SEQ ID No 1.

| | PET depolymerization yield after 24 |
|---|---|
| Variants | hours compared to SEQ ID No 1 (%) |
| V73: A24R | 107% |
| V74: F187I | 163% |

The variants of Table 4 have the exact amino acid sequence of SEQ ID No 1 except the substitution listed in Table 4.

### Degrading Activity Under Alkaline Condition as Compared to the Esterase of SEQ ID No 1

The specific degrading activity and/or PET depolymerization yield after 24 hours of esterases used according to the invention were also measured at pH 8 as exposed in Example 2.1 (or in Example 2.2 for variants V16 and V19). The activity was measured at 65° C. except for the variant V16 for which the activity was measured at 68° C., and variant V74 measured at 60° C. The specific degrading activity and/or PET depolymerization yield after 24 hours of the esterase of SEQ TD No 1 is used as a reference and considered as 100% specific degrading activity and/or 1000% PET depolymerization yield after 24 hours.

Variants showing an increased specific degrading activity at pH 8 compared to esterase of SEQ TD No 1 are further shown in Table 5 below.

TABLE 5
Specific degrading activity of the esterases used according
to the invention at pH 8, compared to SEQ ID No 1

| | Relative specific |
|---|---|
| | degrading activity |
| | compared to SEQ |
| Variants | ID No 1 at pH 8 (%) |
| V4: F208T | 130% |
| V5: F208L | 133% |
| V7: F208S | 112% |
| V9: F208I | 156% |
| V10: F208A | 143% |
| V11: F208R | 121% |
| V12: F208N | 114% |
| V13: F208G | 111% |
| V14: Q182E | 111% |
| V19: F208I + D203C + S248C + | 132% |
| V170I + Y92G + N213P + Q182E | |
| V16: F208M + D203C + S248C + | 199% |
| V170I + Y92G + N213P + Q182E | |
| V19: F208I + D203C + S248C + | 132% |
| V170I + Y92G+ N213P + Q182E | |
| V70: F208M + D203C + S248C + | 223 % |
| V170I + Y92G + N213P + | |
| Q182E + S13L + D158E | |
| V74: F187I | 113% |

Variants listed in Table 5 have the exact amino acid sequence as set forth in SEQ ID Ni, except the combination of substitutions listed in Table 5, respectively.

Variants showing an increased PET depolymerization yield at pH 8 compared to esterase of SEQ TD No 1 are further shown in Table 6 below.

TABLE 6
PET depolymerization yield of the esterase of the invention
after 24 hours at pH 8 and 60° C. compared to SEQ ID No 1.

| | Relative specific degrading activity |
|---|---|
| Variants | compared to SEQ ID No 1 at pH 8 (%) |
| V74: F187I | 113% |

### Degrading Activity Under Acidic Conditions and Basic Conditions, as Compared to the Esterase of SEQ ID No 1

An improvement ratio was calculated between the relative specific degrading activity at pH 5.2 and/or PET depolymerization yield after 24 hours at pH 5.2 and the relative specific degrading activity at pH 8 and/or PET depolymerization yield after 24 hours at pH 8. The variants showing an improvement ratio above 1.1, i.e. showing a higher increase of activity in acidic conditions rather than in basic conditions (as compared to SEQ TD No 1) are listed in Table 7 below.

TABLE 7
Variants showing a higher increase of activity
in acidic conditions rather than in basic
conditions, as compared to SEQ ID No 1

| | Improvement ratio |
|---|---|
| | (relative activity at pH 5.2/ |
| Variants | relative activity at pH 8) |
| V1: N211D | 3.69 |
| V2: N204G | 1.17 |
| V4: F208T | 1.40 |
| V5: F208L | 1.32 |
| V6: F208M | 1.88 |
| V7: F208S | 1.38 |
| V8: F208Q | 1.43 |
| V11: F208R | 1.14 |
| V14: Q182E | 1.34 |
| V15: Q182D | 1.33 |
| V16: F208M + D203C + | 2.24 |
| S248C + V170I + Y92G + | |
| N213P + Q182E | |
| V19: F208I + D203C + | 3.55 |
| S248C + V170I + Y92G + | |
| N213P + Q182E | |
| V70: F208M + D203C + | 3.24 |
| S248C + V170I + Y92G + | |
| N213P + Q182E + S13L+ | |
| D158E | |
| V73: A24R | 1.57 |
| V74: F187I | 1.44 |

Variants have the exact amino acid sequence as set forth in SEQ ID No 1, except the substitution or combination of substitutions listed in Table 7, respectively. Said variants exhibit a higher increase of activity in acidic conditions, than in basic conditions, as compared to SEQ ID No 1.

### Example 3—Evaluation of the Thermostability of Esterases of the Invention

The thermostability of esterases of the invention has been determined and compared to the thermostability of the esterase of SEQ ID No 1.

Different methodologies have been used to estimate thermostability:
(1) Circular dichroism of proteins in solution;
(2) Residual esterase activity after protein incubation in given conditions of temperatures, times and buffers;
(3) Residual polyester's depolymerization activity after protein incubation in given conditions of temperatures, times and buffers;
(4) Ability to degrade a solid polyester compound (such as PET or PBAT or analogues) dispersed in an agar plate, after protein incubation in given conditions of temperatures, times and buffers;
(5) Ability to perform multiple rounds of polyester's depolymerization assays in given conditions of temperatures, buffers, protein concentrations and polyester concentrations;
(6) Differential Scanning Fluorimetry (DSF); Details on the protocol of such methods are given below.

### 3.1 Circular Dichroism

Circular dichroism (CD) has been performed with a Jasco 815 device (Easton, USA) to compare the melting temperature (Tₘ) of the esterase of SEQ ID No 1 with the Tm of the esterases of the invention. Technically 400 μL protein sample was prepared at 0.5 mg/mL in defined condition of pH (Talon buffer pH 8, sodium acetate buffer 100 mM pH 5 or 5.2) and used for CD. A first scan from 280 to 190 nm was realized to determine two maxima intensities of CD corresponding to the correct folding of the protein. A second scan was then performed from 25° C. to 110° C., at length waves corresponding to such maximal intensities and providing specific curves (sigmoid 3 parameters y=a/(1+e\hat{ }((x−x0)/b))) that were analyzed by Sigmaplot version 11.0 software, the Tm is determined when x=x0. The Tm obtained reflects the thermostability of the given protein. The higher the Tm is, the more stable the variant is at high temperature.

### 3.2 Residual Esterase Activity

1 mL of a solution of 40 mg/L (in Talon buffer or 0.2 M sodium acetate buffer pH 5.0 or 0.2 M sodium acetate buffer pH 5.2 or sodium acetate buffer pH 6.0) of the esterase of SEQ ID No 1 or of an esterase of the invention was incubated at different temperatures (40, 50, 60, 65, 70, 75, 80 and 90° C.) up to 10 days. Regularly, a sample, was taken, diluted 1 to 500 times in a 0.1 M potassium phosphate buffer pH 8.0 or 0.2 M sodium acetate buffer pH 5.0 or 0.2 M sodium acetate buffer pH 5.2 or sodium acetate buffer pH 6.0 and para nitro phenol-butyrate (pNP-B) assay was realized. 20 μL of sample are mixed with 175 μL of 0.1M potassium phosphate buffer pH 8.0 or 0.2 M sodium acetate buffer pH 5.0 or 0.2 M sodium acetate buffer pH 5.2 or sodium acetate buffer pH 6.0 and 5 μL of pNP-B solution in 2-methyl-2 butanol (40 mM). Enzymatic reaction was performed at 30° C. under agitation, for 15 minutes and absorbance at 405 nm was acquired by microplate spectrophotometer (Versamax, Molecular Devices, Sunnyvale, CA, USA). Activity of pNP-B hydrolysis (initial velocity expressed in μmol of pNPB/min) was determined using a standard curve prepared in the same conditions of buffer and pH than the enzymatic assay for the liberated para nitro phenol in the linear part of the hydrolysis curve.

### 3.3 Residual Polyester Depolymerizing Activity

10 mL of a solution of 40 mg/L (in Talon buffer or 0.2 M sodium acetate buffer pH 5.0 or 0.2 M sodium acetate buffer pH 5.2 or sodium acetate buffer pH 6.0) of the esterase of SEQ ID No 1 and of an esterase of the invention respectively were incubated at different temperatures (40° C., 50° C., 60° C., 65° C., 70° C., 75° C., 80° C. and 90° C.) up to 30 days. Regularly, a 1 mL sample was taken, and transferred into a bottle containing 100 mg of amorphous PET (prepared according to WO 2017/198786 to reach a crystallinity below 20%) micronized at 250-500 μm and 49 mL of 0.1M potassium phosphate buffer pH 8.0 or 0.2 M sodium acetate buffer pH 5.0 or 0.2 M sodium acetate buffer pH 5.2 or sodium acetate buffer pH 6.0 and incubated at 50° C., 55° C., 60° C., 65° C. or 70° C. 150 μL of buffer were sampled regularly. When required, samples were diluted in 0.1 M potassium phosphate buffer pH 8. Then, 150 μL of methanol and 6.5 μL of HCl 6 N were added to 150 μL of sample or dilution. After mixing and filtering on 0.45 μm syringe filter, samples were loaded on UHPLC to monitor the liberation of terephthalic acid (TA), MHET and BHET. Chromatography system used was an Ultimate 3000 UHPLC system (Thermo Fisher Scientific, Inc. Waltham, MA, USA) including a pump module, an autosampler, a column oven thermostated at 25° C., and an UV detector at 240 nm. The column used was a Discovery® HS C18 HPLC Column (150×4.6 mm, 5 μm, equipped with precolumn, Supelco, Bellefonte, USA). TA, MHET and BHET were separated using a gradient of MeOH (30% to 90%) in 1 mM of H₂SO₄ at 1 mL/min. Injection was 20 μL of sample. TA, MHET and BHET were measured according to standard curves prepared from commercial TA and BHET and in house synthetized MHET in the same conditions than samples. Activity of PET hydrolysis (μmol of PET hydrolysed/min or mg of equivalent TA produced/hour) was determined in the linear part of the hydrolysis curve, such curve being set up by samplings performed at different time during the first 24 hours. Equivalent TA corresponds to the sum of TA measured and of TA contained in measured MHET and BHET.

### 3.4 Degradation of a Polyester Under Solid Form

1 mL of a solution of 40 mg/L (in Talon buffer or potassium phosphate buffer 0.1M pH 8.0 or citrate phosphate buffer 0.1M pH 6.0 or sodium acetate buffer 0.1M pH 5.2) of the esterase of SEQ ID No 1 and of an esterase of the invention respectively were incubated at different temperatures (40° C., 50° C., 60° C., 65° C., 70° C., 75° C., 80° C. and 90° C.) up to 30 days. Regularly, enzyme preparation was sampled and deposited in a well created in an agar plate containing PET. Preparation of agar plates was realized by solubilizing 50 mg of PET in hexafluoro-2-propanol (HFIP) and pouring this medium in a 250 mL aqueous solution. After HFIP evaporation at 50° C. under 140 mbar, the solution was mixed with potassium phosphate buffer pH 8.0 or with citrate phosphate buffer pH 6.0 or with sodium acetate buffer pH 5.2 to obtain a final concentration of 0.5 mg/mL of PET and 0.1 M of buffer containing 1% agar. Around 30 mL of the mixture is used to prepare each plate and stored at 4° C. 1 μL, 5 μL or 20 μL of enzyme preparation was deposited in a well created in an agar plate containing PET at pH 8.0, 6.0 or 5.2, respectively.

The diameter or the surface area of the halos formed due to the polyester degradation by wild-type esterase and variants of the invention were measured and compared after 2 to 24 hours at 50° C., 55° C., 60° C., 65° C. or 70° C. The half-life of the enzyme at a given temperature corresponds to the time required to decrease by a 2-fold factor the diameter of the halo.

### 3.5 Multiple Rounds of Polyester's Depolymerization

The ability of the esterase to perform successive rounds of polyester's depolymerization assays was evaluated in an enzymatic reactor. A Minibio 500 bioreactor (Applikon Biotechnology B.V., Delft, The Netherlands) was started with 3 g of amorphous PET (prepared according to WO 2017/198786 to reach a crystallinity below 20%) and 100 mL of 100 mM sodium acetate buffer pH 5.0 or 100 mM sodium acetate buffer pH 5.2 or 100 mM sodium acetate buffer pH 6.0 containing 3 mg of esterase. Agitation was set at 250 rpm using a marine impeller. Bioreactor was thermostated at 50° C., 55° C., 60° C., 65° C. or 70° C. by immersion in an external water bath. pH was regulated at 5.0 or 5.2 or 6.0 by addition of NaOH at 3 M. The different parameters (pH, temperature, agitation, addition of base) were monitored thanks to BioXpert software V2.95. 1.8 g of amorphous PET (prepared according to WO 2017/198786 to reach a crystallinity below 20%) were added every 20 h. 500 μL of reaction medium was sampled regularly.

Amount of TA, MHET and BHET was determined by HPLC, as described in example 2.3. Amount of EG was determined using an Aminex HPX-87K column (Bio-Rad Laboratories, Inc, Hercules, California, United States) thermostated at 65° C. Eluent was K₂HPO₄ 5 mM at 0.6 mL·min⁻¹. Injection was 20 μL. Ethylene glycol was monitored using refractometer.

The percentages of hydrolysis were calculated based on the ratio of molar concentration at a given time (TA+MHET+BHET) versus the total amount of TA contained in the initial sample, or based on the ratio of molar concentration at a given time (EG+MHET+2×BHET) versus the total amount of EG contained in the initial sample. Rate of degradation is calculated in mg of total liberated TA per hour or in mg of total EG per hour.

Half-life of enzyme was evaluated as the incubation time required to obtain a loss of 50% of the degradation rate.

### 3.6 Differential Scanning Fluorimetry (DSF)

DSF was used to evaluate the thermostability of the wild-type protein (SEQ ID No 1) and variants thereof by determining their melting temperature (Tm), temperature at which half of the protein population is unfolded. To estimate Tm values at pH 8.0, protein samples were prepared at a concentration of 6.25 μM in buffer A consisting of 20 mM Tris HCl pH 8.0, 300 mM NaCl. To estimate Tm values at pH 5.2, protein samples were first prepared at a concentration of 25 μM in buffer B consisting in potassium phosphate buffer, 100 mM pH 8.0. 6 μL of prepared protein sample were subsequently diluted with 18 μL of buffer C consisting of sodium acetate, 100 mM pH 5.09 to reach a final pH value of 5.2 containing 6,25p M of diluted protein sample. The SYPRO orange dye 5000x stock solution in DMSO was first diluted to 250× in water. Protein samples were loaded onto a white clear 96-well PCR plate (Bio-Rad cat #HSP9601) with each well containing a final volume of 25 μl. The final concentration of protein and SYPRO Orange dye in each well were 6 μM (0.14 mg/ml) and 10× respectively. The PCR plates were then sealed with optical quality sealing tape and spun at 2000 rpm for 1 min at room temperature. DSF experiments were then carried out using a CFX96 real-time PCR system set to use the 450/490 excitation and 560/580 emission filters. The samples were heated from 25 to 100° C. at the rate of 0.3° C./second. A single fluorescence measurement was taken every 0.03 second. Melting temperatures were determined from the peak(s) of the first derivatives of the melting curve using the Bio-Rad CFX Manager software.

Esterase of SEQ ID No 1 and esterases of the invention were then compared based on their Tm values. At pH 5.2, a ΔTm of 0.8° C. was considered as significant to compare variants inside a same set of experiments. Tm values correspond to the average of at least 3 measurements.

### Results

### Thermostability as Compared to the Esterase of SEQ ID No 1 Under Acidic Conditions

The gains of Tm of esterase variants used according to the invention, as compared to the esterase of SEQ ID No 1, are shown in Table 8 below, as evaluated according to Example 3.6 at pH 5.2.

TABLE 8
Tm of the variants used in the invention compared
to Tm of the esterase of SEQ ID No 1 at pH 5.2

| | Δ Tm (° C.) |
|---|---|
| Variants | pH 5.2 |
| V1: N211D | +2.7° C. |
| V2: N204G | +1.6° C. |
| V19: F208I + D203C + S248C + | +14.8° C. |
| V170I + Y92G + N213P + Q182E | |
| V16: F208M + D203C + S248C + | +16.2° C. |
| V170I + Y92G + N213P + Q182E | |
| V17: F208T + D203C + S248C + | +16.8° C. |
| V170I + Y92G + N213P + Q182E | |
| V18: F208L + D203C + S248C + | +15.5° C. |
| V170I + Y92G + N213P + Q182E | |
| V70: F208M + D203C + S248C + | +16.3° C. |
| V170I + Y92G + N213P + Q182E + | |
| S13L + D158E | |
| V71: F208M + D203C + S248C + | +15.3° C. |
| V170I + Y92G + N213P + Q182E + S13L | |
| V72: F208M + D203C + S248C + V170I + | +16.4° C. |
| Y92G + N213P + Q182E + D158E | |

The variants listed in table 8 have the exact amino acid sequence as set forth in SEQ ID No 1, except the substitution or combination of substitutions listed in Table 8, respectively.

### Thermostability Under Acidic and Alkaline Conditions as Compared to the Esterase of SEQ ID No 1

Table 9 below shows the increase of thermostability of the variants used in the invention compared to reference esterase of SEQ ID No 1 at pH 5.2, and at pH 8 evaluated according to Example 3.6.

TABLE 9
Gains of increase of thermostability of the variants used in the
invention compared to esterase of SEQ ID No 1 at pH 5.2 and at pH 8

| | Δ Tm | Δ Tm |
|---|---|---|
| | (° C.) | (° C.) |
| Variant | pH 5.2 | pH 8 |
| V19: F208I + D203C + S248C + | +14.8° C. | +11.1° C. |
| V170I + Y92G + N213P + Q182E | | |
| V16: F208M + D203C + S248C + | +16.2° C. | +14.1° C. |
| V170I + Y92G + N213P + Q182E | | |
| V17: F208T + D203C + S248C + | +16.8° C. | +14.9° C. |
| V170I + Y92G + N213P + Q182E | | |
| V18: F208L + D203C + S248C + | +15.5° C. | +14.4° C. |
| V170I + Y92G + N213P + Q182E | | |
| V70: F208M + D203C + S248C + | +16.3° C. | +15.3° C. |
| V170I + Y92G + N213P + Q182E + | | |
| S13L + D158E | | |
| V71: F208M + D203C + S248C + | +15.3° C. | +13.9° C. |
| V170I + Y92G + N213P + Q182E + | | |
| S13L | | |
| V72: F208M + D203C + S248C + | +16.4° C. | +15.3° C. |
| V170I + Y92G + N213P + Q182E + | | |
| D158E | | |

Variants listed in Table 9 have the exact amino acid sequence as set forth in SEQ ID No 1, except the combination of substitutions listed in Table 9, respectively.

Said variants exhibit a higher increase of thermostability in acidic conditions, than in basic conditions, as compared to SEQ TD No 1.

1-26. (canceled)

27. A process for degrading a polyester or a polyester containing material, wherein the process comprises a step of depolymerization of said polyester performed by contacting the polyester or a polyester containing material in a reaction medium at a pH between 3 and 6 with an esterase able to degrade said polyester wherein said esterase (i) has at least 90% identity to the full length amino acid sequence set forth in SEQ ID NO: 1, (ii) contains at least one substitution selected from F208M/W/G/N/R/I/A/Q/L/S/T/E, T11E/M, R12D/N/Q/E/F, S13E, A14E/D, T16E, A17T, T61S/V, A62D, F90A/Y, Y92G/D, W155A, T157S, P179D/E, Q182D/E, F187Y/I, D203C/K/R, N204D/E/G, A205D, S206D/E, N211D/Y/E, S212F, N213P/D, N214D, A215N, S218A, V219I, Y220M/F, Q237D, F238E, N241E/D, N243E/D, L247T, V170I, G135A, V167Q/T, S248C and A24R, as compared to the amino acid sequence SEQ ID NO: 1, wherein the positions are numbered by reference to the amino acid sequence set forth in SEQ ID NO: 1 and (iii) has a polyester degrading activity at a pH between 3 and 6.

28. The process according to claim 27, wherein said esterase comprises at least one substitution and/or combination of substitutions selected from N211D, N204G, F208T/M/L/S/Q/R, Q182D/E, A24R, F187I, F208M/I/T/L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L, F208M+D203C+S248C+V170I+Y92G+N213P+Q182E+D158E.

29. The process according to claim 28, wherein the depolymerization step is implemented at a pH between 4 and 6.

30. The process according to claim 27, wherein the depolymerization step is implemented at a temperature between 50° C. and 72° C.

31. The process according to claim 27, wherein the polyester is selected from polyethylene terephthalate (PET), polytrimethylene terephthalate (PTT), polybutylene terephthalate (PBT), polyethylene isosorbide terephthalate (PEIT), polybutylene adipate terephthalate (PBAT), polycyclohexylenedimethylene terephthalate (PCT), glycosylated polyethylene terephthalate (PETG), poly (butylene succinate-co-terephtalate) (PBST), poly(butylene succinate/terephthalate/isophthalate)-co-(lactate) (PBSTIL), “polyolefin-like” polyesters and blends/mixtures of these polymers.

32. The process according to claim 27, wherein the esterase comprises at least one substitution selected from F208M/W/G/N/R/I/A/Q/L/S/T/E, T11E/M, R12D/N/Q/E/F, S13E, A14E/D, T16E, T61S/V, A62D, F90A/Y, Y92G/D, W155A, T157S, Q182D/E, D203C/K, N204E/G, S206D, N211D/Y/E, S212F, N213P, V219I, Y220M, Q237D, N241E/D, N243E, L247T, V170I, G135A, V167Q/T, S248C, A24R and F187I, or selected from F208M/W/G/N/R/I/A/Q/L/S/T/E, T11M, R12D/E/F, A14E/D, T61S/V, A62D, F90A/Y, Y92G, W155A, T157S, Q182D/E, D203C/K, N204E/G, N211D/Y/E, S212F, N213P, V219I, Q237D, N241E/D, V170I, G135A, V167Q/T, S248C, A24R and F187I.

33. The process according to claim 27, wherein the esterase comprises at least one substitution selected from:
a) A14E/D, F90A/Y, Y92G/D, Q182D/E, N204D/E/G, F208M/W/G/N/R/I/A/Q/L/S/T/E, N211D/Y/E, N213P/D, V219I, V170I, D203C/K/R, S248C, A24R and F187Y/I;
b) A14E, F90A, Y92G/D, Q182D/E, N204G, F208M/G/N/R/I/A/Q/L/S/T, N211D/E, N213P, V219I, V170I, D203C/K/R, S248C, A24R and F187I;
c) F90A, Y92G, Q182D/E, N204G, F208M/G/N/R/I/A/Q/L/S/T, N211D/E, V170I, D203C/K/R, S248C, N213P, A24R and F187I; or
d) N211D/E, N204G, F208M/R/I/Q/L/S/T, Q182D/E, Y92G, V170I, D203C/K/R, S248C, N213P, A24R and F 187I.

34. The process according to claim 27, wherein the esterase comprises at least one substitution selected from F208M/W/G/N/R/I/A/Q/L/S/T/E, F208M/G/N/R/I/A/Q/L/S/T, F208M/R/I/Q/L/S/T, or F208NMR/Q/L/S/T.

35. The process according to claim 27, wherein the esterase comprises at least a combination of substitutions selected from:
a) F208M/W/G/N/R/I/A/Q/L/S/T/E+D203C+S248C+V170I+Y92G/D+N213P/D+Q182D/E and F208M/W/G/N/R/I/A/Q/L/S/T/E+D203K+V170I+Y92G/D+N213P/D+Q182D/E;
b) F208M/I/L/T+D203C+S248C+V170I+Y92G+N213P+Q182E and F208M/I/L/T+D203K+V170I+Y92G/D+N213P/D+Q182D/E;
c) F208M/I/L/T+D203C+S248C+V170I+Y92G+N213P+Q182E and F208M/I/L/T+D203K+V170I+Y92G+N213P+Q182E; or
d) F208M+D203C+S248C+V170I+Y92G+N213P+Q182E and F208M+D203K+V170I+Y92G+N213P+Q182E.

36. The process according to claim 27, wherein the esterase comprises at least one substitution selected from N211D/E, N204G, F208M/R/Q/L/S/T, Q182D/E, A24R and F187I or N211D, F208M/Q/L/S/T, Q182D/, A24R and F187I, and exhibits a greater increase of polyester degrading activity compared to the enzyme of SEQ ID NO: 1 at a pH between 3 and 6 than at a pH above 7.

37. The process according to claim 27, wherein the esterase comprises at least one combination of substitutions selected from F208M+D203C+S248C+V170I+Y92G+N213P+Q182E, F208I+D203C+S248C+V170I+Y92G+N213P+Q182E and F208M+D203C+S248C+V170I+Y92G+N213P+Q182E+S13L+D158E and exhibits a greater increase of polyester degrading activity compared to the enzyme of SEQ ID NO: 1 at a pH between 3 and 6 than at a pH above 7.

38. The process according to claim 27, wherein the esterase comprises at least one combination of substitutions selected from F208I+D203C+S248C+V170I+Y92G+N213P+Q182E, F208M+D203C+S248C+V170I+Y92G+N213P+Q182E, F208T+D203C+S248C+V170I+Y92G+N213P+Q182E, F208L+D203C+S248C+V170I+Y92G+N213P+Q182E, F208M+D203C+S248C+V170I+Y92G+N213P+Q182E+S13L+D158E, F208M+D203C+S248C+V170I+Y92G+N213P+Q182E+S13L and F208M+D203C+S248C+V170I+Y92G+N213P+Q182E+D158E and exhibits a greater increase of thermostability compared to the enzyme of SEQ ID NO: 1 at a pH between 3 and 6 than at a pH above 7.

39. The process according to claim 27, wherein the esterase comprises at least one combination of substitutions selected from F208M+D203C+S248C+V170I+Y92G+N213P+Q182E, F208I+D203C+S248C+V170I+Y92G+N213P+Q182E and F208M+D203C+S248C+V170I+Y92G+N213P+Q182E+S13L+D158E and exhibits both a greater increase of thermostability and a greater increase of polyester degrading activity compared to the enzyme of SEQ ID NO: 1 at a pH between 3 and 6 than at a pH above 7.

40. The process according to claim 27, wherein the polyester comprises at least a terephthalic acid monomer (TA), or the polyester is polyethylene terephthalate (PET).

41. The process according to claim 27, wherein the process is a process for recycling a polyester or a polyester of a polyester containing material comprising submitting the polyester to a depolymerization step performed at a pH between 3 and 6, and recovering and optionally purifying the monomers and/or oligomers.

42. The process according to claim 27, wherein the esterase has a polyester degrading activity at a pH between 5 and 5.5.

43. The process according to claim 27, wherein the esterase has at least 95% identity with SEQ ID NO: 1.

44. The process according to claim 43, wherein the esterase has a polyester degrading activity at a pH between 5 and 5.5.

45. The process according to claim 28, wherein the esterase has at least 95% identity with SEQ ID NO: 1.


SEQUENCE LISTING (1 sequence)

SEQ ID NO: 1 - protein, 258 aa, synthetic construct
  REGION 1..258: note = LC CUTINASE
SNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ